(19) United States
(12) Patent Application Publication
(10) Pub. No.: US 2005/0003480 A1
(43) Pub. Date: Jan. 6, 2005

(54) April variants and methods thereof

(75) Inventors: John Rudolph Desjarlais, Pasadena, CA (US); Eugene Alexander Zhukovsky, West Hollywood, CA (US)

Correspondence Address: Robin M. Silva, Esq.;Dorsey & Whitney LLP, Intellectual Property Department, Four Embarcadero Center, Suite 3400, San Francisco, CA, 94111-4187, US

(73) Assignee: Xencor

(21) Appl. No.: 10/820,465

(22) Filed: Mar. 31, 2004

Related U.S. Application Data
(63) Continuation-in-part of application No. 10/338,083, filed on Jan. 6, 2003.
(60) Provisional application No. 60/528,104, filed on Dec. 8, 2003.
(60) Provisional application No. 60/523,880, filed on Nov. 20, 2003.
(60) Provisional application No. 60/482,081, filed on Jun. 23, 2003.
(60) Provisional application No. 60/459,097, filed on Mar. 31, 2003.

(51) Int. Cl. C07K 14/705; C07H 21/04; A61K 38/17

(52) U.S. Cl. 435/69.1; 514/12; 530/350; 536/23.5; 435/320.1; 435/325

(57) ABSTRACT

The invention relates to novel proteins of APRIL variants that modulate the biological activity as compared to naturally occurring APRIL or BAFF and nucleic acids encoding these proteins. The invention further relates to the use of the novel proteins in the treatment of BAFF or APRIL-related disorders.

This application claims benefit under 35 U.S.C. §199(e) to U.S. Ser. No. 60/459097, filed Mar. 31, 2003; Ser. No. 60/528,104, filed Dec. 8, 2003; Ser. No. 60/523,880, filed Nov. 20, 2003; Ser. No. 60/482,081, filed Jun. 23, 2003; Ser. No. 60/452,707, filed Mar. 7, 2003; and U.S. Ser. No. 10/338,083 and PCT/US03/00394 filed on Jan. 6, 2003, all of which are expressly incorporated by reference in their entirety.

### FIELD OF THE INVENTION

The invention relates to novel proteins of APRIL which modulate the signaling of naturally occurring APRIL and/or other TNF super family (TNFSF) proteins with dominant negative antagonist activity, agonist activity, receptor antagonist activity (also known as competitive inhibition activity) and nucleic acids encoding these proteins. The invention further relates to the use of the novel proteins in the treatment of APRIL and/or TNFSF related disorders, such as pathologies of the immune system and oncological conditions.

### BACKGROUND OF THE INVENTION

APRIL (also known as TRDL-1 alpha, TALL-2, TNFSF13) and it's closest homologue, BAFF (also known as B-cell Activation Factor, BLyS, TALL-1, THANK, zTNF4 and TNFSF13B) are members of the TNF super family (TNFSF) of proteins. The prototype of the family, Tumor Necrosis Factor Alpha (TNFA ), originally discovered for its in vivo effect causing tumors to regress, is a key mediator of inflammation. BAFF and APRIL proteins participate in a variety of cellular and intracellular signaling processes and are synthesized as a type 2 membrane proteins and fold into conserved β-pleated sheet structures. It is known that BAFF and APRIL form homotrimers typical of the TNFSF. In addition, BAFF and APRIL also form heterotrimers together.

The receptors for TNFSF members also represent a family of structurally related molecules, including at least 26 receptors and/or receptor decoy molecules. The extracellular domains of members of this family are composed of multiple repeats of a cysteine-rich domain (CRD), a small protein domain containing six conserved cysteines that form three disulfide bonds. The intracellular domains of these receptors are more diverse, although many members of the family contain a death domain that mediates apoptosis and other receptor signaling events. These members are all capable of inducing apoptosis via interaction with one or more intracellular adaptor molecules that also contain death domains. Other signaling receptors of this family signal via interactions with a family of adaptor molecules called TRAFs (TNF receptor associated factors). Signaling through APRIL receptors (for example, TACI and BMCA) is triggered by binding of an oligomeric (and for the most part, trimeric) APRIL ligand.

The three-dimensional structures of BAFF and APRIL are made up of a sandwich of two anti-parallel beta-sheets with the “jelly roll” or Greek key topology and it assembles into a trimeric complex. The cognate receptors of BAFF and APRIL are part of the related TNFSF of receptors. Furthermore, there appears to be significant conservation of the mode of receptor binding. In general, each receptor monomer binds within the cleft formed between two of the ligand monomers. The overall similarity in tertiary and quaternary structures of both the ligands and their complexes with receptors indicates that well-proven strategies for inhibition or agonism of one ligand-receptor system may be transferable to the other proteins in the family. Thus the present invention provides methods for the creation of variants of APRIL that are modulators of BAFF, APRIL and/or TNFSF signaling pathways.

Lymphocyte populations are regulated by replication and death. B-cell maturation relies on signals through both BCR (B-cell receptor) and survival co-stimulation by BAFF. Therefore, BAFF is important for survival of B-cells and humoral immune response. Normally, only a small number of B-cells mature due to a vigorous selection directed at selecting of a population of B-cells that are not auto reactive. Overexpression of BAFF in transgenic (Tg) animals results in the state of immune hyperreactivity, which is manifested in an increase of peripheral B-cell lymphocyte counts, breakdown of B-cell tolerance (inappropriate survival of autoreactive lymphocytes), and enlarged lymphoid organs and spleen. All this is accompanied by the appearance of anti-DNA antibodies, an increase in antibody secretion (e.g., IgM, IgG and IgA), and Ig-deposition in the kidneys resulting in glomerulonephritis that leads to autoimmune-like syndromes similar to systemic lupus erythematosus (SLE), Sjogren syndrome (SS), and the like. It has also been observed that SLE, RA, and SS patients showed a correlation of high BAFF concentration with elevated levels of anti-dsDNA antibody a biochemical marker of these diseases. It has been shown that in RA patients, concentration of BAFF in synovial fluids is much higher than in blood.

BAFF also stimulates T-cells to a much lesser degree and increases the population of activated effector T-cells. There are three known receptors of BAFF: BAFF-R, TACI, and BCMA. The first one is exclusively specific to BAFF and the latter two are shared with APRIL. Phenotypes of BAFF knockout mice (KO) and BAFF-R mutation strain of mice (A/WySnJ) suggest that BAFF-R is the main receptor for BAFF and is responsible for control of B-cell maturation. TACI controls B-cell homeostasis and T-cell Independent immune response and appears to act as an inhibitory BAFF receptor. The role of BCMA is unclear thus far.

The therapeutic interest in APRIL as a drug target lies in its strict specificity so that attenuation of the function of this cytokine may result in no widespread side effects. Animal studies have shown that administration of BAFF-blocking reagents in BAFF transgenic mice reduced diseases incidence and its severity pointing to this cytokine as a crucial mediator of these autoimmune diseases. For example, TACI-Fc inhibited the development of proteinuria and prolonged survival of NZB/WF1 mice (the main SLE model in mice).

APRIL is expressed in the same type of cells as BAFF, that is peripheral blood lymphocytes (PBL) and monocytes/macrophages. Similar to BAFF, APRIL also co-stimulates (together with BCR) B-cell proliferation and IgM production. APRIL KO mice die in utero suggesting that it may play an important role in development. BCMA preferentially binds to APRIL over BAFF.

APRIL stimulates colon cancer cells that express no TACI or BCMA (i.e. neither of the known APRIL receptors). In a similar assay, BAFF has no effect on tumor cells. Also, s-BCMA, which can bind and block APRIL, inhibited cancer cell growth. All these facts taken together suggest the existence of a specific APRIL-R that has not been identified yet.

A need still exists for proteins that can interfere with intracellular signaling processes. Thus, it is an object of the present invention to provide proteins comprising APRIL variants with receptor-interaction domains that are modified such that each domain has significantly reduced affinity and/or signaling capacity for one or more cognate receptor(s). Such modified domains preferably retain association with individual monomer domains of naturally occurring BAFF or APRIL, but exhibit a dominant-negative phenotype, antagonizing the action of related naturally occurring domains via their sequestration into inactive oligomeric complexes. In another embodiment, APRIL homotrimers or homodimers may bind to one or more cognate receptors, including but not limited to BCMA and TACI, and inactivate or reduce one or more intracellular signaling processes. In a further embodiment, APRIL variant homo-oligomers may act as agonists or superantagonists of one or more cognate receptors.

### SUMMARY OF THE INVENTION

In accordance with the objects outlined above, the present invention provides variants of extracellular domains of APRIL proteins that modulate the bioactivity of BAFF or APRIL, particularly the signaling of naturally occurring BAFF and/or APRIL proteins. In general, the APRIL variant comprises at least one amino acid substitution at a position selected from the group consisting of: 131, 132, 133, 138, 139, 140, 143, 144, 146, 148, 151, 160, 170, 171, 172, 173, 174, 175, 176, 177, 178, 179, 180, 181, 186, 187, 188, 189, 190, 192, 194, 195, 196, 197, 198, 200, 202, 205, 206, 207, 208, 228, 230, 231, 232, 233, 234, 235, 236, 237, 238, 239, 240, 241, 242, 243, 244, 245, 246, 247, 248, 249, 250, 271, 273, 275, 276, 278, 282, 284, and 285. Of these, preferred positions are within the Large Domain, the Small Domain and/or the DE loop, including 138, 139, 140, 170, 171, 172, 173, 174, 175, 176, 177, 178, 180, 181, 186, 187, 188, 189, 195, 196, 197, 198, 205, 206, 207, 208, 230, 231, 232, 233, 234, 235, 236, 237, 238, 239, and 240. Generally preferred positions are 138, 139, 140, 170, 171, 172, 173, 174, 175, 176, 177, 178, 180, 181, 186, 187, 188, 189, 195, 196, 197, 198, 205, 206, 207, 208, 230, 231, 232, 233, 234, 235, 236, 237, 238, 239, and 240. Particularly preferred positions are: 138, 139, 140, 170, 171, 172, 173, 174, 205, 205, 206, 207, 208, 230, 231, 232, 233, 234, 235, 236, 237, 238, 239, and 240, with positions 175, 176, 177, 178, 179, 180, 181, 195, 196, 197, and 198 being especially preferred. Exceptionally preferred are modifications are positions 175, 176, 177, 178, 179, 180, 181, 195, 196, 197, and 198, with ultimately preferred positions including 186, 187, 188, and 189.

The present invention provides variant APRIL proteins comprising an amino acid sequence that have at least one modification as compared to the naturally occurring APRIL protein sequence. In a preferred embodiment, variant APRIL proteins will physically interact with a naturally occurring BAFF or APRIL protein to form mixed oligomers substantially incapable of activating receptor signaling.

In another embodiment, variant APRIL proteins of the invention are in at least monomeric form and comprise amino acid sequences that have at least one modification as compared to a naturally occurring APRIL protein sequence, wherein said variant APRIL proteins will interact with a receptor interface at at least one receptor binding site to render said receptor substantially incapable of activating receptor signaling.

Preferably, a variant APRIL protein of the present invention has at least one receptor contact domain that has reduced affinity for a desired receptor as compared to its corresponding wild-type APRIL protein and retains the ability to interact with other receptor interaction domains.

More specifically, variant APRIL proteins of the present invention physically interact with a naturally occurring BAFF or APRIL protein to reduce the ability of the naturally occurring protein to activate at least one receptor. Variant APRIL proteins of the present invention may interact with other members of the TNFSF.

More specifically, variant APRIL proteins comprise at least one modified receptor-contact domain that has reduced affinity and/or signaling capacity for a desired receptor wherein said protein cannot substantially activate the desired receptor, but retains the ability to interact with other BAFF or APRIL proteins.

In a preferred embodiment, variant APRIL proteins have reduced affinity and/or signaling capacity for one or more receptors while maintaining or increasing signaling through one or more alternative receptors. For example, variant APRIL proteins that have reduced signaling through BCMA while maintaining signaling through TACI are expected to be potent antagonists of APRIL and/or BAFF-mediated biology.

The present invention relates to the use of variants of APRIL for the inhibition or treatment of a variety of diseases. The variants are specifically engineered to modulate their biological signaling capacity, while retaining their ability to function as oligomeric species, either in complex with themselves or with naturally occurring members of the TNFSF.

In a preferred embodiment, variant APRIL proteins are engineered to yield significantly reduced signaling through APRIL and BAFF receptors compared to wild-type APRIL proteins while maintaining affinity for other BAFF or APRIL proteins to allow formation of mixed oligomers, most preferably trimers. Such variant APRIL proteins are referred to as “dominant negative APRIL variants” or “DN-APRIL”. The dominant negative APRIL variants act by sequestering one or more naturally occurring BAFF or APRIL proteins in heterotrimers that are incapable of appreciably activating biological signaling through one or more cognate receptors. Consequently, DN-APRIL proteins act to antagonize the action of naturally occurring BAFF and/or APRIL. In some cases, both variant APRIL and variant BAFF proteins can be used (see U.S. Ser. No. 10/______, filed Mar. 5, 2004 and entitled BAFF VARIANTS AND METHODS THEREOF, hereby incorporated by reference.

In another embodiment, variant APRIL proteins are engineered to act as agonists. These agonist variants may find use in treating a variety of immune deficiency syndromes, including but not limited to common variable immunodeficiency (CVID) and immunoglobulin-A (IgA) deficiency. Additionally, conditions or diseases requiring elevated immune response or an increased number of B-cells, elevated concentrations of immunoglobulins would benefit from APRIL agonism (e.g. AIDS, cancer, infections, etc.).

It is a further embodiment to provide APRIL variants that provide a reduction in agonism comprising the following more preferred positions

In a further embodiment, variant APRIL proteins, in monomer or dimer form, are engineered to bind to a receptor but to reduce or eliminate signaling of an APRIL and/or BAFF receptor.

In yet another embodiment, the APRIL variants of the present invention may be used as an experimental tool to identify molecules that interact with it in the signal transduction pathway associated with APRIL.

The present invention provides non-naturally occurring variant APRIL proteins (e.g. proteins not found in nature) comprising amino acid sequences with at least one modification compared to the wild-type APRIL proteins.

Preferred embodiments utilize variant APRIL proteins that interact with one or more wild-type TNFSF members to form mixed oligomers incapable of substantially activating receptor signaling. Preferably, variant APRIL proteins with at least one amino acid change are used as compared to a wild-type APRIL protein.

In another preferred embodiment, modifications may be made either individually or in combination, with any combination being possible. Preferred embodiments utilize at least one, and preferably more, positions in each variant APRIL protein. For example, amino acid substitutions may be combined to form double variants or triple point variants.

In a further embodiment, an APRIL molecule may be chemically modified, for example by PEGylation, phosphorylation or glycosylation.

In another aspect, portions of the N- or C-termini may be deleted. In a further embodiment, an APRIL molecule may be circularly permuted.

In an additional aspect, the two or more extracellular domains of the variant APRIL proteins are covalently linked by a linker peptide or by other means. Preferably, the linker peptide is a sequence of at least one and not more than about 30 amino acid residues and comprises one or more of the following amino acid residues: Gly, Ser, Ala, or Thr.

In a further aspect, the invention provides recombinant nucleic acids encoding the non-naturally occurring variant APRIL proteins, expression vectors, and host cells.

In an additional aspect, the invention provides methods of producing a non-naturally occurring variant APRIL protein comprising culturing the host cell of the invention under conditions suitable for expression of the nucleic acid.

In a further aspect, the invention provides pharmaceutical compositions comprising a variant APRIL protein of the invention and a pharmaceutical carrier.

In a further aspect, the invention provides methods for treating an APRIL and/or BAFF related disorder comprising administering a variant APRIL protein of the invention to a patient.

Although it is known that APRIL does not bind with high affinity to BAFF-R, it remains possible that in certain contexts, APRIL can indeed interact with BAFF-R. For example, if APRIL monomers are mixed with BAFF monomers to generate mixed trimers (containing APRIL and BAFF monomers in 1:2 or 2:1 ratios), BAFF-R binding may occur. It is therefore an object of the present invention to provide non-naturally occurring variants of APRIL that have reduced BAFF-R binding. In a further object, the variants comprise modifications at at least one of the following positions: 176, 241, 197, 231, 175, 230, 195, 173, 192, 181, 179, 133, 131, 233 and 177. Of these, modifications at one or more of 173, 175, 192, 195, 197, 230, and 231 are particularly preferred. Particular modifications include at least one modification selected from the group consisting of D173E, D173N, D173S, T175A, T175D, T175N, T175S, T192Q, R195D, R195E, R195K, I197A, I197E, I197Q, I197T, I197Y, P230A, P230D, P230N, R231A, R231H, R231K, AND R231L.

It is an object of the present invention to provide non-naturally occurring variants of APRIL that have increased BAFF-R binding. In a further object, the variants comprise modifications at at least one of the following positions: 133, 206, 232, 197, 202, 132, 181, 235, 208, 131, 173. Particularly preferred positions are 173 and 197, with one or modifications of D173K, I197L and I197V.

It is an object of the present invention to provide non-naturally occurring variants of APRIL that have decreased TACI binding. In a further object, the variants comprise modifications at at least one of the following positions: 176, 241, 197, 175, 231, 173, 195, 230, 174, 181, 192, 133, 206, 177, 179, 131. Particularly preferred positions include 173, 175, 192, 195, 197, 230 and 231. Particularly preferred modifications include at least one of D173E, D173G, D173N, D173S, T175A, T175D, T175K, T175N, T175S, T192K, T192Q, R195D, R195E, R195K, I197A, I197E, I197Q, I197T, I197Y, P230A, P230D, P230N, R231A, R231H, R231K, and R231L.

It is an object of the present invention to provide non-naturally occurring variants of APRIL that have increased TACI binding. In a further object, the variants comprise modifications at at least one of the following positions: 133, 206, 232, 181, 202, 197, 131, 173, 208, and 132. Particularly preferred positions included 173 and 197, and particularly preferred modifications include D173 and I197V.

It is an object of the present invention to provide non-naturally occurring variants of APRIL that have decreased BCMA binding. In a further object, the variants comprise modifications at at least one of the following positions: 232, 241, 176, 197, 206, 231, 173, 175, 230, 195, 133, 192, 174, 181, 179, 131, 177, 237, 208. Particularly preferred positions are 173, 175, 192, 195, 197, 230 and 231. Particularly preferred modifications include D173E, D173G, D173N, D173S, T175A, T175D, T175K, T175S, T192K, T192Q, T192V, R195D, R195E, R195K, I197A, I197E, I197Q, I197T, I197Y, P230A, P230D, P230N, R231A, R231H, R231K, and R231L.

It is an object of the present invention to provide non-naturally occurring variants of APRIL that have increased BCMA binding. The variants comprise modifications at at least one of the following positions: 133, 239, 202, 206, 208, 176, 192, 197, 131, 173, 132, and 181. Particularly preferred positions are 173, 192 and 197, and particularly preferred modifications include D173K, T192N, and I197V.

In an additional aspect, the invention provides variant APRIL proteins that exhibit antagonist activity that is either dominant negative antagonism or receptor antagonism. The variants comprise modifications at at least one of the following positions: 143, 144, 146, 148, 172, 174, 192, 194, 196, 198, 200, 206, 207, 228, 230-237, 240-250, 271, 273, 275, 276, 278, 282, 284, and 285.

### BRIEF DESCRIPTION OF THE DRAWINGS

FIG. 1 illustrates a general mechanism by which a dominant negative APRIL protein may antagonize the action of a naturally occurring BAFF, APRIL or other TNFSF protein. Ovals represent an APRIL protein monomer and triangles represent receptor molecules. Naturally occurring APRIL or BAFF proteins typically signal by organizing receptors into an active complex. Variant APRIL proteins are modified (bumps and sticks) within their receptor contact domains such that receptor binding and/or signaling is reduced. When variant APRIL trimers are incubated with naturally occurring TNFSF protein trimers, they equilibrate to form four different trimer species, three of which are inactive. At sufficient concentrations of dominant-negative variant APRIL, essentially all of the naturally occurring TNFSF protein will be sequestered into inactive heterotrimeric complexes. Through this and related mechanisms of inactivating TNFSF proteins, the dominant-negative APRIL variants will exert their therapeutic effects.

FIG. 2 depicts the experimentally determined structure of BAFF, a homolog of APRIL.

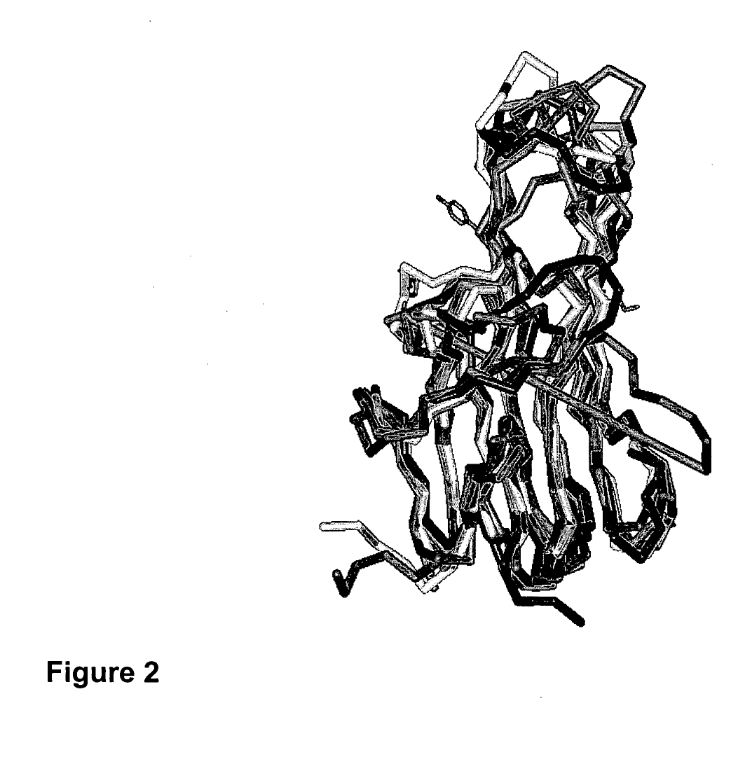

FIG. 3 shows a Multiple Sequence Alignment (MSA) of human TNFSF members. FIG. 3 also shows position numberings of each individual sequence. For TNF-α (TNFA) and TNFB (LT-α), the numbering is based on current convention. For APRIL, the numbering is based on the full-length precursor sequence of the protein. For sequences in which a structure of the ligand-receptor complex has been determined experimentally, residues that lie at the ligand-receptor interface are highlighted in gray. These interfaces, highlighted in black, are used to define 7 general receptor contact regions of the TNFSF ligands. A generic numbering system, beginning with position number 1, is also included above the MSA for reference.

### DETAILED DESCRIPTION OF THE INVENTION

The present invention is directed to novel proteins that modulate the biological activity of BAFF, APRIL or other TNFSF proteins

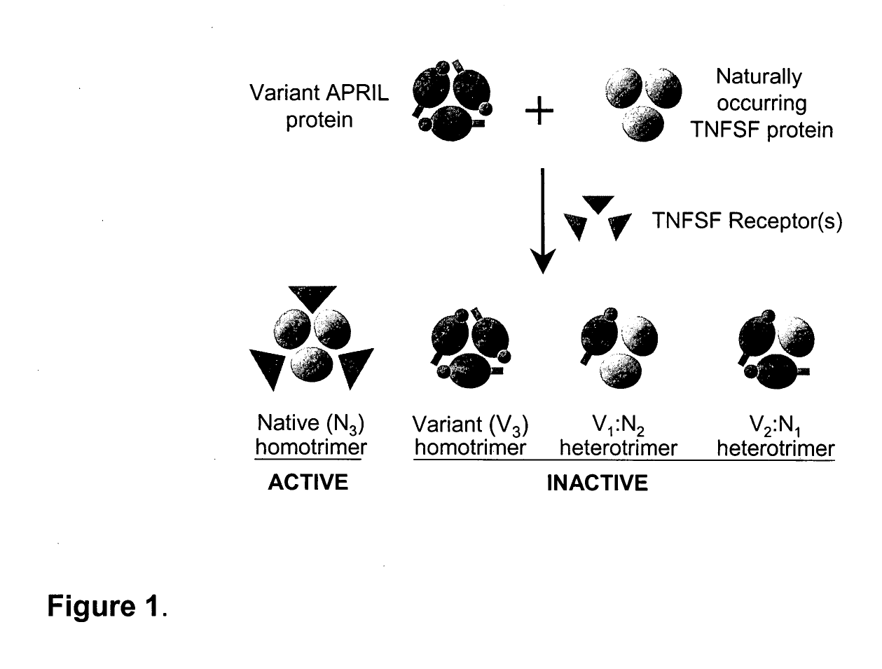

The present invention is directed to novel proteins that exhibit a dominant negative phenotype or mechanism of action. The dominant-negative therapeutic strategy is based on the design of novel APRIL variants that have reduced receptor binding and/or activation properties as compared to naturally occurring APRIL or BAFF proteins, and the ability to oligomerize with naturally occurring BAFF or APRIL proteins (FIG. 1). In other words, APRIL variants that do not substantially activate at least one APRIL or BAFF receptor (as compared to a naturally occurring APRIL protein) will exchange with at least one naturally occurring BAFF or APRIL protein and sequester it into inactive hetero-oligomers or partially inactive hetero-oligomers, inhibiting at least one biological activity. However, other mechanisms of action are also possible. Thus APRIL variants of the present invention have at least one altered biological activity, which can include alteration of agonist activity, antagonist activity, receptor binding, and specificity of receptor binding.

The APRIL variant proteins of the present invention may be designed by modifying APRIL proteins at key receptor contact points in order to modify the ability of the ligand bind to and/or to activate receptor. In preferred embodiments, the exchange and physical interaction of these oligomeric APRIL variants with naturally occurring BAFF or APRIL proteins results in the complete or partial deactivation of the naturally occurring proteins. To help accomplish this goal more effectively, the APRIL variants can also be designed to facilitate hetero-oligomerization with naturally occurring BAFF or APRIL proteins.

Accordingly, the present invention is directed to variant APRIL proteins (sometimes referred to herein as “non-naturally occurring APRIL protein”) that have at least one biological property of a naturally occurring APRIL or BAFF protein that is altered or modulated. “Altered” or “modulated” in this context includes both increases and decreases in activity. “APRIL or BAFF biological activity” or grammatical equivalents thereof include, but are not limited to, modulation (either increases or decreased) of either agonist or antagonist activity, altered receptor binding (including, but not limited to, binding to APRIL and/or BAFF receptors (BAFF-R, TACI, or BCMA), or altered receptor specificity binding (e.g. decreased binding to BCMA and no significant change in binding (e.g. binding to BCMA and no significant change in binding to TACI; increased binding to BCMA, and no significant change in binding to TACI; etc.).

In a preferred embodiment, the variant APRIL proteins of the invention are antagonists of naturally occurring BAFF or APRIL proteins. By “antagonists of naturally occurring BAFF or APRIL” herein is meant that the variant APRIL protein inhibits or significantly decreases the biological activity, particularly the activation of receptor signaling by a naturally occurring member of the BAFF or APRIL protein. In the context of the invention, a “significant decrease” or a “significant increase” in activity comprises at least a 10% change, with changes of about 20%-50% and up to 90-100% being included.

In a preferred embodiment, the variant APRIL protein physically interacts with its naturally occurring APRIL or BAFF protein such that the complex comprising the variant APRIL and wild-type BAFF or APRIL is incapable of activating one or more APRIL or BAFF receptors, or causes a significant decrease in activation. Preferably, the variant APRIL protein preferentially interacts with a wild-type BAFF or APRIL to form mixed trimers with the wild-type proteins such that receptor binding does not occur and/or APRIL or BAFF signaling through at least one receptor is not initiated, or, again, either is significantly decreased (FIG. 1).

In an alternative embodiment, the variant APRIL protein physically interacts with a naturally occurring BAFF protein. Because BAFF is known to form heterotrimers with APRIL, dominant-negative variant APRIL protein may be used to inhibit naturally occurring APRIL or BAFF proteins.

By “extracellular domain” or “ECD” as used herein is meant the segment of protein existing predominantly outside the cell. For transmembrane proteins, this segment can be tethered to the cell through a transmembrane domain or released from the cell through proteolytic digestion. Alternatively, the extracellular domain could comprise the whole protein or amino acid segments thereof when secreted from the cell. In general, APRIL is expressed as type II transmembrane proteins (extracellular C terminus). The unprocessed protein generally contains an atypical signal anchor/intracellular domain of about 10 to 80 amino acids. The extracellular region may be about 140-220 amino acids in length. Soluble forms of APRIL proteins may result from proteolytic cleavage of the signal polypeptide by a furin family protease or directly by recombinant methods.

Unless otherwise disclosed, the variant APRIL proteins of the present invention are composed of the extracellular domain or functional equivalents thereof. That is, the variants of the present invention do not comprise transmembrane domains unless specifically noted. In certain embodiments of the present invention, the variant APRIL proteins would antagonize the membrane bound naturally occurring form of a BAFF or APRIL protein and in other embodiments, the variant APRIL proteins would antagonize the soluble form of a naturally occurring BAFF or APRIL protein. However, it should be noted that in some cases, for example in gene therapy applications, the entire protein, including the transmembrane domain and/or the intracellular domain.

By “nonconservative” modification herein is meant a modification in which the wild type residue and the mutant residue differ significantly in one or more physical properties, including hydrophobicity, charge, size, and shape. For example, modifications from a polar residue to a nonpolar residue or vice-versa, modifications from positively charged residues to negatively charged residues or vice versa, and modifications from large residues to small residues or vice versa are nonconservative modifications. Although not required, in a preferred embodiment, the variant APRIL proteins of the present invention have at least one nonconservative modification of a natural, non-natural or synthetic amino acid.

Modifications of the present invention may include those to surface, boundary and core areas of an APRIL protein. See, for example, U.S. Pat. Nos. 6,188,965 and 6,269,312, hereby incorporated by reference. In another preferred embodiment, modifications may be made to surface residues, as these generally contribute the most to binding interactions. In addition, as outlined below, the variants may include additional mutations that confer desirable properties such as stability, or may include compensatory mutations that allow, for example, a variant that exhibits desired oligomerization capabilities but also exhibits decreased stability to become more stable.

The variant proteins may be generated, for example, by using a system previously described in U.S. Pat. Nos. 6,188,965; 6,296,312; 6,403,312; and 6,708,120; U.S. Ser. Nos. 09/419,351, 09/782,004, 09/927,790, 09/877,695, and 09/877,695; alanine scanning (see U.S. Pat. No. 5,506,107), gene shuffling ((WO 01/25277), site saturation mutagenesis, mean field, sequence homology, or other methods known to those skilled in the art that guide the selection of point mutation sites and types.

In a preferred embodiment, sequence and/or structural alignments may be used to generate the variant APRIL proteins of the invention. As is known in the art, there are a number of sequence-based alignment programs; including for example, Smith-Waterman searches, Needleman-Wunsch, Double Affine Smith-Waterman, frame search, Gribskov/GCG profile search, Gribskov/GCG profile scan, profile frame search, Bucher generalized profiles, Hidden Markov models, Hframe, Double Frame, Blast, Psi-Blast, Clustal, and GeneWise. There are also a wide variety of structural alignment programs known. See for example VAST from the NCBI (ncbi.nlm.nih.gov:80/Structure/VAST/vast. shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9): 739-747. (1998)); (Orengo et al., Structure 5(8): 1093-108 (1997); Dali (Holm et al., Nucleic Acid Res. 26(1): 316-9 (1998), all of which are incorporated by reference).

The methods of the present invention can be applied to any naturally occurring APRIL protein or related system in which individual domains oligomerize to form an active complex. These domains may be modified in a number of ways to remove or reduce receptor binding and/or activation. In addition, each modified domain may be covalently coupled to at least one additional modified domain to generate dominant negative proteins with enhanced antagonistic activity.

As illustrated in FIG. 1, a variant APRIL protein is preferably modified such that interactions with at least one receptor molecule are altered. Preferably, these modifications would not substantially affect the ability of the variant domain to interact with and sequester naturally occurring BAFF or APRIL proteins. In a preferred embodiment, these modifications may be combined with additional modifications that enhance the ability of variant APRIL proteins to hetero-oligomerize with one or more naturally occurring TNFSF proteins, including but not limited to BAFF and APRIL. Most preferably, modifications that affect receptor activation and oligomerization are also combined with chemical modifications (e.g., glycosylation, phosphorylation, PEGylation, fusions, etc.) that improve pharmacokinetic properties. More preferably, the present invention is also directed to novel proteins and nucleic acids possessing APRIL antagonist activity.

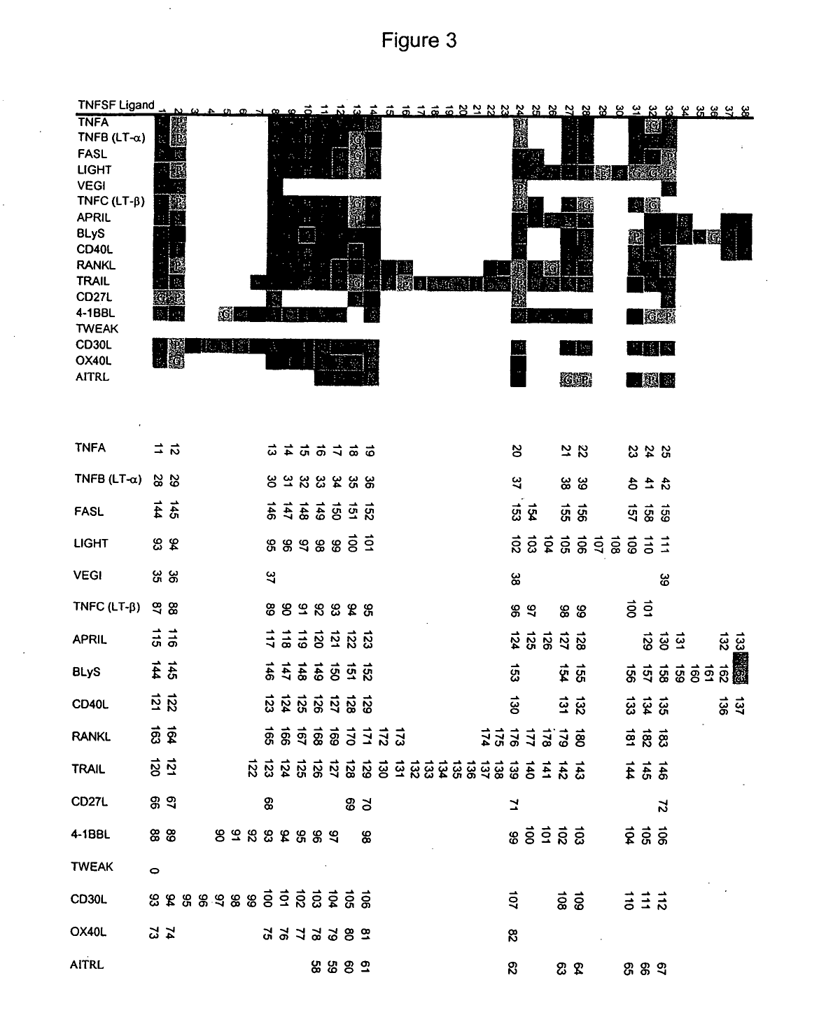

The variant APRIL proteins and nucleic acids of the invention are distinguishable from naturally occurring or wild-type APRIL. By “naturally occurring”, “wild-type”, “native”, or grammatical equivalents, herein is meant an amino acid sequence or a nucleotide sequence that is found in nature and includes allelic variations; that is, an amino acid sequence or a nucleotide sequence that usually has not been intentionally modified. Accordingly, by “non-naturally occurring” or “synthetic” or “recombinant” or grammatical equivalents thereof, herein is meant an amino acid sequence or a nucleotide sequence that is not found in nature; that is, an amino acid sequence or a nucleotide sequence that usually has been intentionally modified. Representative amino acid sequences of naturally occurring human TNFSF members are shown in FIG. 3. It should be noted, that unless otherwise stated, all positional numbering of variant APRIL proteins and variant APRIL nucleic acids is based on these sequences. That is, as will be appreciated by those in the art, an alignment of TNFSF proteins may be done using standard programs, as is outlined below, with the identification of “equivalent” positions between proteins within the alignment. Thus, the variant APRIL proteins and nucleic acids of the invention are non-naturally occurring; that is, they do not exist in nature.

By “dominant negative” phenotype or “mechanism of action” herein is meant a protein comprising at least one APRIL domain or monomer that has reduced affinity and/or altered signaling for a desired receptor or receptors such that the protein cannot substantially interact and/or signal with the desired receptor or receptors, but retains the ability to oligomerize with other receptor interaction domains (see FIG. 1). In some embodiments, the oligomeric proteins also retain the ability to interact with and/or signal through other receptors. Depending on the composition of the oligomeric ligand complex, i.e., 2 variants:1 native or 1 variant:2 native heterotrimers, the degree to which ligand mediated receptor activation is inhibited will vary (see FIG. 1). In other words, receptor activation may be completely inhibited in a complex comprising 2variant:1 native, whereas activation may be reduced in complexes comprising other ratios of variant: native. See also Menart, V., et al., (2000) Eur. J Physiol., 439, R113-R115; U.S. Patent Pub. Nos. 2002/0039588, 2002/0040132, 2002/0037286, 2002/0037280; all of which are incorporated herein by reference. Monte Carlo simulations of heterotrimer assembly, as a function of the relative concentration of variant APRIL to naturally occurring BAFF or APRIL, show that, in general, greater than 99% of the naturally occurring APRIL or BAFF monomers will be sequestered when a 10-fold excess of variant APRIL monomer is added.

As will be appreciated by those of skill in the art, two general approaches for creating the dominant negative variants of the present invention include: (1) modifying individual receptor interaction domains to reduce or eliminate receptor binding and/or signaling; and, (2) covalently coupling modified receptor interaction domains to enhance inhibition of receptor activation.

In a preferred embodiment, individual APRIL proteins are modified within their receptor contact domains to reduce or eliminate receptor binding and/or signaling. For example, amino acid substitutions can be generated as modifications in the receptor contact domains that reduce or eliminate receptor binding. See U.S. Pat. No. 5,506,107; U.S. Ser. Nos. 09/798,789; 09/981,289; 10/262,630; 60/374,035; U.S. Ser. No. 10/338,785 and PCT/US03/00393, filed on Jan. 6, 2003 and U.S. Ser. No. 10/338083 and PCT/US03/00394, filed Jan. 6, 2003, all of which are hereby incorporated by reference.

Preferable modifications (e.g. substitutions, insertions, deletions, etc.) that affect receptor binding or signaling may be identified using a variety of techniques, including structural alignment methods, sequence alignment methods, etc., as described above. In many cases, the amino acids in the APRIL ligand that interact with the receptor can be identified directly from a three-dimensional structure or model of the APRIL ligand-receptor complex or computer modeling.

As is known in the art, there are a number of sequence alignment methodologies that may be used. For example, sequence homology based alignment methods may be used to create sequence alignments of TNFSF members (Altschul et al., J. Mol. Biol. 215(3): 403-410 (1990); Altschul et al., Nucleic Acids Res. 25:3389-3402 (1997), both incorporated by reference). These sequence alignments are then examined to determine the observed sequence variations. These sequence variations may be tabulated to define a set of variant APRIL proteins.

Sequence based alignments may be used in a variety of ways. For example, a number of related proteins may be aligned, as is known in the art, and the “variable” and “conserved” residues defined; that is, the residues that vary or remain identical between the family members can be defined. The extent of variability at positions within the alignment can also be used to identify positions in the protein where mutation is permissive regarding preservation of structural features of the protein. These results may be used to guide the design of variant protein libraries whose properties can be probed experimentally. For example, the positions of high variability between family members (i.e. low conservation) may be randomized, either using all or a subset of amino acids. Alternatively, the sequence variations may be tabulated and appropriate substitutions defined from them. Alternatively, the allowed sequence variations may be used to define the amino acids considered at each position during a computational modeling and/or screening process. Another variation is to bias the score for amino acids that occur in the sequence alignment, thereby increasing the likelihood that they are found during computational screening but still allowing consideration of other amino acids. This bias would result in a focused library of variant APRIL proteins but would not eliminate from consideration amino acids not found in the alignment.

In a preferred embodiment, as highlighted in FIG. 3, the amino acid sequences of TNFSF proteins may be aligned into a multiple sequence alignment (MSA). The alignment shown in FIG. 3 was derived originally from the Pfam database, and then further manipulated according to structural alignment (using CE) of the crystal structures of TNFA, TNFB, BAFF and APRIL. The MSA may also be used to extend the known structural information for additional recognized TNFSF members and other structural homologues and families. Due to the high extent of structural homology between APRIL and BAFF (as well as other TNFSF members), the MSA may be used as a reliable predictor of the effects of modifications at various positions within the alignment. For this, the TNFA sequence and numbering shown in FIG. 3 can be used as an MSA reference point for any other TNFSF protein sequence, such as APRIL. As used herein, referral to “APRIL protein positions corresponding to TNFA amino acid X”, represents referral to a collection of equivalent positions in APRIL and structural homologues and families. For example, APRIL protein positions corresponding to TNFA amino acid L75 correspond to APRIL:T177.

For example, analysis of a structure of the complex of TNFB with the p55 (R1) receptor indicates that the amino acid Q118 in TNFB directly contacts the receptor. The analogous residue A226 from TRAIL also directly contacts the DR5 receptor. The MSA thus predicts that the analogous residue 197 from TNFA also contacts a receptor. Consistent with this prediction, mutation of TNFA-197 to R or T results in a significant loss of receptor-binding affinity and biological signaling activity. The analysis for this contact position can be extended to APRIL, predicting that APRIL: I197 is important for receptor interactions. This kind of analysis may be performed for all receptor contact regions of the ligands.

FIG. 3 highlights 7 canonical receptor contact regions based on analysis of known structures and mutational data. In preferred embodiments of the invention, each of the 7 regions highlighted in FIG. 3 as a receptor-contact region is used to define modification sites for the creation of variants of APRIL. In additional preferred embodiments, such modifications alter receptor affinity and/or signaling capacity. In additional preferred embodiments, these modifications also preserve the ability of each protein to oligomerize with naturally occurring BAFF or APRIL proteins.

Using the alignment system depicted in FIG. 3 or other alignment programs discussed above, one can use as a reference point, the numbering system of any alignment program and may correlate the relevant positions of the TNFA protein with equivalent positions in APRIL or structural homologues and families.

For purposes of the present invention, the areas of APRIL proteins to be modified are selected from the group consisting of the Large Domain, Small Domain, the DE loop, and the trimer interface. The Large Domain and the Small Domain are two separate receptor contact domains, each made up of several non-contiguous linear segments of the protein. Hence, substitution of amino acids at these positions is expected to alter the ability of APRIL proteins to interact and/or signal with one or more APRIL or BAFF receptors. The trimer interface mediates interactions between individual APRIL protein monomers and between BAFF and APRIL monomers. Trimerization positions can be identified either directly from the crystal structure of the APRIL protein, or by analogy to another TNFSF protein. In a preferred embodiment, positions from one APRIL protein monomer containing atoms that are within 5 angstroms distance from a neighboring monomer are designated as trimer interface positions. DE loop modifications have been shown to affect expression and solubility properties of other TNFSF proteins. Hence, substitution of amino acids at DE loop positions is expected to alter the ability of APRIL proteins to form higher order structures. Modifications may be made solely in one of these areas or in any combination of these and other areas.

In a preferred embodiment, inspection of the MSA and the three-dimensional structure of BAFF indicate that the Large Domain preferred positions to be modified in APRIL include but are not limited to TNFA corresponding positions 28-34, 63-69, 112-115, and 137-147 that is, APRIL positions 138-140, 170-174, 205-208, and 230-240. For the Small Domain, the preferred positions to be modified include but are not limited to TNFA corresponding positions 72-79 and 95-98, that is APRIL positions 175-181 and 195-198. For the DE Loop, the preferred positions to be modified include but are not limited to TNFA corresponding positions 84-89, that is, APRIL positions 186-190.

In a preferred embodiment, the choice of modification site and type is made by referring to other sequences in the alignment. Thus, in a preferred embodiment, the original amino acid X from sequence A is mutated to amino acid Y from sequence B, such that Y is a nonconservative substitution relative to amino acid X. For example, the amino acid Y87 from TNFA aligns with the non-conservative R189 from APRIL. Indeed, as previous studies have shown, the Y87R substitution in TNFA leads to a significant decrease in receptor binding and signaling by TNFA. In additional embodiments, more conservative mutations can also be utilized. In additional embodiments, the wild-type residue is mutated to alanine.

In a preferred embodiment, the choice of modification site and type is made by referring to APRIL's closest homolog BAFF. As described in U.S. Ser. No. 10/______, filed Mar. 5, 2004 and entitled BAFF VARIANTS AND METHODS THEREOF, hereby incorporated by reference, a large number of BAFF variants were characterized with respect to their affect on interactions with BAFF-R, TACI, and BCMA, as well as they're ability to modulate the agonist activity of BAFF. APRIL is a close homolog of BAFF, and, like BAFF, interacts with BCMA and TACI. The interactions of APRIL with BCMA and TACI are expected to be similar, if not identical to, the interactions of BAFF with BCMA and TACI. Consistent with this model, many substitutions in BAFF that enhance interactions with BCMA and TACI are substitutions to the amino acid observed at the equivalent position of APRIL. For example, the substitutions S162D, Y206F, and L211V in BAFF enhance binding to BCMA, consistent with the fact that the equivalent amino acids in APRIL are D132, F176, and V181, respectively. Because of the homology between BAFF and APRIL, we anticipate that positions in BAFF that are important for receptor binding will also be important in its homolog APRIL. As used herein, use of the phrase “by analogy to BAFF” refers to the application of this reasoning. For example, substitutions at Y206 in BAFF (e.g. Y206E) have an impact on binding to BCMA. By analogy to BAFF, substitutions at the equivalent (defined according to the MSA) amino acid F176 in APRIL are also expected to have an impact on binding to BCMA.

BAFF positions which have been discovered to influence binding to receptors include the following: Q159, S162, Q163, D203, K204, T205, Y206, A207, L211, T228, R231, I233, E238, L240, N242, P264, R265, E266, N267, Q269, S271, D273, and D275. Modifications at these positions have been demonstrated to lead to an increase or decrease in binding to at least one of the receptors BAFF-R, TACI, or BCMA. The equivalent amino acids in APRIL are S131, D132, S133, D173, V174, T175, F176, T177, V181, T192, R195, I197, S202, R206, Y208, P230, R231, A232, R233, K235, N237, S239, and H241, respectively. By analogy to BAFF, modifications at these positions are all expected to lead to an increase or decrease in binding to at least one of APRIL's receptors.

For cases in which the native amino acids of BAFF and APRIL at an equivalent position are identical (or highly similar), one expects that substitutions in BAFF that impacted receptor interactions will have virtually identical effects when made in APRIL. Such positions include the following BAFF/APRIL amino acid pairings: D203/D173, T205/T175, T228/T192, R231/R195, I233/I197, P264/P230, and R265/R231, and Y206/F176. For example, the substitutions T228K and I233E in BAFF reduce binding to all of BAFF's receptors. The same substitutions at the equivalent positions in APRIL, T192K and I197E, are therefore also expected to reduce binding to all of APRIL's receptors. As used herein, the phrase “by equivalence to BAFF” refers to the application of this reasoning.

In a preferred embodiment, useful modifications at receptor contact and/or trimerization interfaces are selected using protein design or modeling algorithms such as PROTEIN DESIGN AUTOMATION® (PDA®) technology (see, U.S. Pat. Nos. 6,188,965; 6,269,312; 6,403,312; and 6,708,120; U.S. Ser. Nos. 09/714,357; 09/812,034; 09/827,960; 09/837,886; 09/782,004 and 10/218,102, all hereby incorporated by reference). As is known in the art, algorithms in this class generally use atomic-level or amino acid level scoring functions to evaluate the compatibility of amino acid sequences with the overall tertiary and quaternary structure of a protein. Thus, algorithms of this class can be used to select receptor-binding disruptions that do not substantially perturb the ability of variant APRIL proteins to properly fold and oligomerize with themselves or their naturally occurring targets. These technologies typically use high-resolution structural information of the target protein as input. In a preferred embodiment, an experimentally determined structure of the APRIL protein is used as input. In additional preferred embodiments, models of APRIL/receptor complexes may be used as structural scaffolds to guide the design of variant APRIL ligands that possess reduced receptor binding and/or signaling and/or dominant-negative activity. In additional embodiments, comparative analysis of various APRIL/receptor complexes may guide the design of variants that have selective binding and/or signaling through APRIL or BAFF receptors.

In alternative embodiments, protein design algorithms may be used to generate mutations in individual receptor interaction domains that create steric repulsion between the receptor interaction domain and the receptor. Other mutations that may be generated include, but are not limited to, mutations that create electrostatic repulsion, and mutations that create unfavorable desolvation of amino acids.

Similarly, molecular dynamics calculations may be used to computationally screen sequences by individually calculating mutant sequence scores and compiling a list.

In a preferred embodiment, residue pair potentials may be used to score sequences (Miyazawa et al, Macromolecules 183): 534-552 (1985), expressly incorporated by reference) during computational screening.

In a preferred embodiment, substitutions, insertions, deletions or other modifications at multiple receptor interaction and/or trimerization domains may be combined. Such combinations are frequently advantageous in that they have additive or synergistic effects on activity. Examples include, but are not limited to, simultaneous substitution of amino acids at the large and small domains (e.g. APRIL positions 173 and 197) large domain and DE loop (e.g. APRIL positions 206 and 190), large domain and trimerization domain (e.g. APRIL positions 239 and 164), or multiple substitutions within a single domain. Additional examples include any and all combinations of substitutions.

In preferred embodiments, the defined receptor contact regions constitute sites for insertion, deletion, or substitution of amino acid residues, or sites for the introduction of chemical modification sites. In a preferred embodiment, deletions or insertions are made in accordance with the MSA.

In additional embodiments, the variants described above can be combined with other modifications to the APRIL protein. These include, but are not limited to, additional amino acid substitutions, insertions, or deletions, and/or chemical (e.g. PEGylation) or posttranslational modifications such as phosphorylation or glycosylation (see WO 99/45026; WO 01/49830; WO 01/49830; WO 02/02597; WO 01/58493; WO 01/51510, U.S. Pat. Nos. 4,002,531; 5,183,550; 5,089,261; 6,153,265; 5,264,209; 5,383,657; 5,766,897; 5,986,068; 4,280,953; 5,089,261; 5,990,237; 6,461,802; 6,495,659; 6,448,369; 6,437,025; 5,900,461; 6,413,507; 5,446,090; 5,672,662; 5,919,455; 6,113,906; 5,985,236; 6,214,966; 6,258,351; 5,932,462; EP 0786 257; EP 0 902 085; EP 1 064 951; EP 0 544 826; EP 0 424 405; EP 0 400 472; EP 0 311 589; Veronese, F. M. (2001) Biomaterials, 22: 405471; all of which are incorporated herein by reference).

In a preferred embodiment, additional amino acid substitutions are made to optimize hetero-oligomer interactions between variant APRIL and its endogenous counterpart and/or to destabilize the oligomeric state of the variant alone. For example, an L57F mutation in TNFA has been designed in order to promote the formation of variant: native heterotrimers while disfavoring the formation of variant homotrimers. Such modifications are useful for promoting the exchange of variant monomers with native monomers in order to promote a dominant-negative mechanism of action. Because APRIL contains an L at this position naturally, the mutation L164F might be expected to affect trimerization with BAFF, APRIL, or both.

Furthermore, protein design methods, outlined herein, may identify compensatory mutations. For example, if a given first mutation that is introduced to alter oligomerization or receptor binding or activation also decreases stability or activity, protein design methods may be used to find one or more additional mutations that serve to recover stability and activity while retaining the altered property. Similarly, protein design methods may identify sets of two or more mutations that together confer the desired property and retained activity and stability, even in cases where one or more of the mutations, in isolation, fails to confer desired properties.

As will be understood by those in the art, variant APRIL proteins which have altered signaling capacity can be discovered by a large variety of methods, including, but not limited to, directed evolution (e.g. error prone PCR, DNA shuffling, etc.), single-site saturation mutagenesis, and alanine-scanning mutagenesis. Furthermore, it is possible that use of these or other methods will allow the discovery of substitutions, insertions, or deletions—which alter receptor binding and/or signaling activity—that lie outside of the 7 canonical contact regions described herein.

In another embodiment, coiled-coil motifs are used to assist dimer assembly (see Dahiyat et al., Protein Science 6:1333-7 (1997) and U.S. Ser. No. 09/502,984; both of which are incorporated herein by reference in their entirety). Coiled coil motifs comprise, but is not limited to one of the following sequences: RMEKLEQKVKELLRKNERLEEEVERLKQLVGER, based on the structure of GCN4; AALESEVSALESEVASLESEVAAL, and LAAVKSKLSAVKSKLASVKSKLAA, coiled-coil leucine zipper regions defined previously (see Martin et al., EMBO J. 13(22): 5303-5309 (1994), incorporated by reference). Other coiled coil sequences from e.g. leucine zipper containing proteins are known in the art and are used in this invention. See, for example, Myszka et al., Biochem. 33:2362-2373 (1994), hereby incorporated by reference).

As will be appreciated by those in the art, additional APRIL proteins may be identified and added to the MSA highlighted in FIG. 3. The source of the sequences may vary widely, and include taking sequences from one or more of the known databases, including, but not limited to, GenBank (.ncbi.nlm.nih.gov/).

In addition, sequences from these databases may be subjected to contiguous analysis or gene prediction; see Wheeler, et al., Nucleic Acids Res 28(1):10-14. (2000) and Burge and Karlin, J Mol Biol 268(1):78-94. (1997).

As used herein variant APRIL or APRIL proteins include APRIL monomers, dimmers or trimers. As is outlined herein, the multimers may also comprise a single variant monomer and wild-type or variant TNFSF monomers, including BAFF and variant BAFF monomers.

The APRIL proteins may be from any number of organisms, with APRIL proteins from mammals being particularly preferred. Suitable mammals include, but are not limited to, rodents (rats, mice, hamsters, guinea pigs, etc.), primates, farm animals (including sheep, goats, pigs, cows, horses, etc); and in the most preferred embodiment, from humans. As will be appreciated by those in the art, APRIL proteins based on APRIL proteins from mammals other than humans may find use in animal models of human disease.

By “heterotrimers (or mixed trimers)” herein is meant that monomers of native and variant APRIL proteins interact to form trimeric APRIL. Heterotrimers or mixed trimers may comprise 1 variant APRIL protein: 2 native APRIL and/or BAFF proteins, 2 variant APRIL proteins: 1 native BAFF or APRIL protein. Similarly, other TNFSF monomers and variant monomers can be included. In some embodiments, heterotrimers or mixed trimers may be formed comprising only variant APRIL proteins. In alternative embodiments, for example, heterotrimers or mixed trimers may comprise 1 variant BAFF protein, 1 native BAFF protein, and 1 APRIL protein; or 2 BAFF proteins: 1 native APRIL protein; and any other possible combinations thereof. In a preferred embodiment, amino acid substitutions, deletions, or insertions that influence the kinetics of exchange between variant and wild type monomers are made either individually or in combination. These substitutions can also be combined with additional substitutions that affect receptor interaction or other properties. By analogy to substitutions that enhance exchange in BAFF, substitutions that have an effect on exchange properties of APRIL may include substitutions at positions T175, F176, T192, I197, R206, S239, and H241, among others.

In a preferred embodiment, the variant APRIL antagonist proteins of the invention are highly specific antagonists for the corresponding wild-type APRIL or BAFF protein. However, in alternative embodiments, the variant APRIL antagonistic proteins of the invention are highly specific for more than one wild-type TNFSF protein. For example, variant APRIL proteins may be specific antagonist of wild-type APRIL only, wild-type APRIL and BAFF, or wild-type BAFF only. Additional characteristics of the variant APRIL antagonist proteins include improved stability, pharmacokinetics, and high affinity for native APRIL or BAFF. Variants with higher affinity toward native APRIL or BAFF, respectively, may be generated from variants exhibiting APRIL or BAFF antagonism as outlined above.

In a preferred embodiment, variant APRIL proteins exhibit decreased biological activity as compared to native APRIL or BAFF, including but not limited to, decreased binding to the one or more receptors, decreased activation or otherwise undesired activities that can lead to deleterious side effects. Variant APRIL proteins that exhibit less than 50% biological activity as compared to native are preferred. More preferred are variant APRIL proteins that exhibit less than 25%, even more preferred are variant proteins that exhibit less than 15%, and most preferred are variant APRIIL proteins that exhibit less than 10% of a biological activity of a naturally occurring APRIL or BAFF. Suitable assays include, but are not limited to, APRIL or BAFF receptor binding assays; transcription assays (using reporter constructs; see Stavridi, supra); size exclusion chromatography assays and radiolabeling/immuno-precipitation; see Corcoran et al., supra); and stability assays (including the use of circular dichroism (CD) assays and equilibrium studies; see Mateu, supra); all of which well known in the art and are expressly incorporated by reference.

In one embodiment, at least one property critical for binding affinity of the variant APRIL proteins is altered when compared to the same property of native APRIL and in particular, variant APRIL proteins with altered receptor affinity for at least one receptor are preferred. Also preferred are variant APRIL proteins with altered affinity toward oligomerization to native BAFF and/or APRIL.

Thus, the invention provides variant APRIL proteins with altered binding affinities such that the variant APRIL proteins will preferentially oligomerize with wild-type APRIL or BAFF, but do not substantially agonize one or more APRIL/BAFF receptors. “Preferentially” in this case means that given equal amounts of variant APRIL monomers and wild-type APRIL or BAFF monomers, at least 10% and more preferably at least 25% of the resulting trimers are mixed trimers of variant and wild-type APRIL or BAFF, with at least about 50% being preferred, and at least about 80-90% being particularly preferred. In other words, it is preferable that the variant APRIL proteins of the invention have greater affinity for wild-type APRIL or BAFF protein as compared to other wild-type APRIL or BAFF proteins, respectively. By “do not substantially interact with TNFSF or APRIL/BAFF receptors” herein is meant that the variant APRIL proteins will not be able to associate with APRIL or BAFF receptors to substantially activate the receptors and/or initiate the APRIL and/or BAFF signaling pathway(s). In a preferred embodiment, at least a 10% decrease in receptor activation is seen, with greater than 20%, 50%, 76%, 80-90% being preferred. By “agonize APRIL or BAFF receptors” herein is meant that a variant APRIL protein enhances the survival co-stimulatory activation of survival, maturation, receptor, etc. signaling.

Non-naturally occurring variants of APRIL that have modulated receptor binding is one aspect of the present invention.

Although it is known that APRIL does not bind with high affinity to BAFF-R, it remains possible that in certain contexts, APRIL can indeed interact with BAFF-R. For example, if APRIL monomers are mixed with BAFF monomers to generate mixed trimers (containing APRIL and BAFF monomers in 1:2 or 2:1 ratios), BAFF-R binding may occur. By analogy to BAFF, APRIL variants with a decrease in BAFF-R binding include but are not limited to at least one of the following positions: 176, 241,197, 231, 175, 230, 195, 173, 192, 181, 179, 133, 131, and 177. More particularly, by equivalence to BAFF, the variants comprise at least one of the following substitutions: D173E, D173N, D173S, T175A, T175D, T175N, T175S, T192Q, T192K, R195D, R195E, R195K, I197A, I197E, I197Q, I197T, I197Y, P230A, P230D, P230N, R231A, R231H, R231K, and R231L.

By analogy to BAFF, variants of APRIL that have increased BAFF-R binding include but are not limited to at least one of the following positions: 133, 206, 232, 197, 202, 132, 181, 235, 208, 131, 173. More preferably, by equivalence to BAFF, at least one of the following substitutions comprises the non-naturally occurring APRIL: D173K, I197L, and I197V.

By analogy to BAFF, variants of APRIL that have decreased TACI binding include but are not limited to at least one of the following positions: 176, 241, 197, 175, 231, 173, 195, 230, 174, 181, 192, 133, 206, 177, 179, and 131. More preferably, by equivalence to BAFF, at least one of the following substitutions comprises the APRIL variant: D173E, D173G, D173N, D173S, T175A, T175D, T175K, T175N, T175S, T192K, T192Q, R195D, R195E, R195K, I197A, I197E, I197Q, I197T, I197Y, P230A, P230D, P230N, R231A, R231H, R231K, and R231L.

By analogy to BAFF, variants of APRIL that have increased TACI binding include but are not limited to at least one of the following positions: 133, 206, 232, 181, 202, 197, 131, 173, 208, 132. More preferably, by equivalence to BAFF, the variants comprise at least one of the following substitutions: D173K, or I197V.

By analogy to BAFF, variants of APRIL that have decreased BCMA binding, include but are not limited to least one of the following positions: 232, 241, 176, 197, 206, 231, 173, 175, 230, 195, 133, 192, 174, 181, 179, 131, 177, 237, 208. More preferably, by equivalence to BAFF, the variants comprise at least one of the following substitutions: D173E, D173G, D173N, D173S, T175A, T175D, T175K, T175S, T192K, T192Q, T192V, R195D, R195E, R195K, I197A, I197E, I197Q, I197T, I197Y, P230A, P230D, P230N, R231A, R231H, R231K, and R231L.

By analogy to BAFF, variants of APRIL that have increased BCMA binding, include but are not limited to at least one of the following positions: 133, 239, 202, 206, 208, 176, 192, 197, 131, 173, 132, 181. Preferably, by equivalence to BAFF, the following substitutions may be used to enhance BCMA binding: D173K, T192N, I197V.

Variant APRIL proteins may be experimentally tested and validated using in vivo and in vitro assays. Suitable assays include, but are not limited to, activity assays and binding assays. Screens that may be utilized in identifying APRIL variants that are antagonists of APRIL or BAFF proteins include, but are not limited to, NF-kB nuclear translocation (Wei et al., Endocrinology 142, 1290-1295, (2001)) or c-Jun (Srivastava et al., JBC 276, 8836-8840 (2001)) transcription factor activation assays, B-cell proliferation assays and IgE secretion assays.

In a preferred embodiment, binding affinities for the following interactions are determined and compared: 1) variant APRIL oligomer formation, 2) wild-type APRIL oligomer formation, 3) variant APRIL binding to cognate receptors (e.g., BCMA and TACI), 4) wild-type APRIL binding to cognate receptors, 5) variant APRIL binding to decoy receptors, and 6) wild-type APRIL binding to decoy receptors. Similar tests can be utilized to determine whether APRIL variants are capable of forming mixed trimers with wild type or variant BAFF proteins. Suitable assays include, but are not limited to, quantitative comparisons comparing kinetic and equilibrium binding constants. The kinetic association rate (Kon) and dissociation rate (Koff), and the equilibrium binding constants (Kd) may be determined using surface plasmon resonance on a BIAcore instrument following the standard procedure in the literature [Pearce et al., Biochemistry 38:81-89 (1999)]. Several alternative methods can also be used to determine binding affinity and kinetics, including but not limited to proximity assays such as AlphaScreen™ (Packard BioScience®) or microcalorimetry (Isothermal Titration Calorimetry, Differential Scanning Calorimetry).

APRIL variants may also be tested to determine whether they'are capable of forming mixed oligomers including but not limited to mixed trimers. In a preferred embodiment, this is accomplished by labeling native APRIL and variant APRIL with distinguishable tags, combining native and variant APRIL, and screening for oligomers that contain both tag types. For example, FLAG-tagged native APRIL and myc-tagged variant APRIL can be combined, and sandwich ELISAs can be performed to identify trimers that contain both FLAG and myc-tag. Another alternative is to run native gels with FLAG-tagged native APRIL and His-tagged variant APRIL to separate the mixture into separate species and detect using coomassie staining or Western blots using both anti-FLAG and anti-His tag antibodies. This method relies on the fact that FLAG and His tags significantly perturb protein migration in native gels. As will be appreciated by those in the art, many alternate protocols could also be used to measure the formation of mixed trimers. Similar tests can be utilized to determine whether APRIL variants are capable of forming mixed trimers with wild type or variant BAFF proteins, or other TNFSF proteins

In a preferred embodiment, variant APRIL proteins have reduced binding and/or signaling through all APRIL and BAFF receptors. Such variant APRIL proteins preferably retain the ability to exchange and physically interact with wild type BAFF or APRIL proteins, such that they are inactivated upon complex formation.

In a preferred embodiment, variant APRIL proteins may only form heterotrimeric complexes with other APRIL proteins. In an alternative embodiment, variant APRIL proteins may only form heterotrimeric complexes with BAFF proteins. As an alternative embodiment, variant APRIL proteins may form heterotrimeric complexes with BAFF and APRIL proteins. As a further embodiment, variant APRIL proteins may form heterotrimeric complexes with at least one TNFSF protein other than BAFF and APRIL, including TNFSF variants such as BAFF variants.

In a preferred embodiment, variant APRIL proteins of the invention have reduced binding and/or signaling through one or more APRIL or BAFF receptors while retaining binding and signaling through other APRIL or BAFF receptors. In a preferred embodiment, APRIL variants have increased binding and/or signaling through TACI. In alternative preferred embodiments, APRIL variants have increased signaling through TACI but decreased signaling through BCMA.

Variant APRIL proteins with altered receptor signaling properties can act either as homotrimers, heterotrimers, or both. For example, a variant APRIL homotrimer with maintained or increased TACI signaling and decreased BCMA signaling may exert its biological activities as a homotrimer that specifically stimulates the inhibitory APRIL/BAFF receptor activity of TACI. Alternatively, a variant APRIL protein homotrimer with maintained or increased TACI signaling and decreased BCMA signaling may exert its biological activities by exchanging and physically interacting with naturally occurring BAFF and/or APRIL proteins to create complexes that stimulate TACI but are incapable of stimulating BAFF-R and/or BCMA.

In a preferred embodiment, variant APRIL proteins can interact with—but not induce signaling through—one or more APRIL/BAFF receptors. These variant APRIL proteins can be used as competitive inhibitors or receptor antagonists, either alone, or as complexes with naturally occurring BAFF or APRIL proteins.

In an alternative preferred embodiment, variant APRIL proteins have increased signaling through one or more APRIL/BAFF receptors and increased or maintained signaling through other APRIL/BAFF receptors. Such variants may find use in research or therapeutic strategies in which stimulation of APRIL or BAFF signaling pathways is desirable. Examples include, but are not limited to B cell proliferation for research purposes, or B cell proliferation for treatment of immune deficiency syndromes or oncological diseases.

In a preferred embodiment, variant APRIL proteins have increased or maintained signaling through BCMA and reduced signaling through TACI, such that stimulation of the APRIL or BAFF pathway is maximized.

Any combinations of the variants disclosed above may be employed to achieve a particular therapeutic result. For example, some combinations include, but are not limited to, at least one variant that increases binding to TACI and decreases binding to BCMA; superagonist activity with TACI (TACI being an inhibitor of BAFF agonism) and minimal binding change for BAFF-R;-optionally an increase in binding to BCMA may also be added. For example, variant APRIL proteins that have reduced signaling through BCMA while maintaining or increasing signaling through TACI are expected to be potent antagonists of APRIL or BAFF-mediated biology.

As outlined above, the invention provides variant APRIL nucleic acids encoding variant APRIL polypeptides. The variant APRIL polypeptide preferably has at least one altered property as compared to the same property of the corresponding naturally occurring APRIL or BAFF polypeptide. The property of the variant APRIL polypeptide is the result of the present invention.

The term “altered property” or grammatical equivalents thereof in the context of a polypeptide, as used herein, further refers to any characteristic or attribute of a polypeptide that can be selected or detected and compared to the corresponding property of a naturally occurring protein. As noted above, preferred altered properties are those of APRIL or BAFF biological activity, as defined herein, including receptor binding (both agonist and antagonist activities), and receptor specificity. In addition, other types of properties include, but are not limited to, proliferation activity (again, a biological property); oxidative stability, substrate specificity, substrate binding or catalytic activity, thermal stability, cytotoxic activity, alkaline stability, pH activity profile, resistance to proteolytic degradation, kinetic association (Kon) and dissociation (Koff) rate, protein folding, inducing an immune response, ability to bind to a ligand, ability to bind to a receptor, ability to be secreted, ability to be displayed on the surface of a cell, ability to oligomerize, ability to signal, ability to stimulate cell proliferation, ability to inhibit cell proliferation, ability to induce apoptosis, ability to be modified by phosphorylation or glycosylation, and the ability to treat disease. Unless otherwise specified, a substantial change in any of the above-listed properties, when comparing the property of a variant APRIL polypeptide to the property of a naturally occurring BAFF or APRIL protein is preferably at least a 20%, more preferably, 50%, more preferably at least a 2-fold increase or decrease. A change in proliferation activity is evidenced by at least a 75% or greater decrease in cell proliferation initiated by a variant APRIL protein as compared to wild-type protein.

A change in binding affinity is evidenced by at least a 5% or greater increase or decrease in binding affinity of APRIL variants to wild-type APRIL receptor proteins or to wild-type BAFF.

In a preferred embodiment, the antigenic profile in the host animal of the variant APRIL protein is similar, and preferably identical, to the antigenic profile of the host APRIL; that is, the variant APRIL protein does not significantly stimulate the host organism (e.g. the patient) to an immune response; that is, any immune response is not clinically relevant and there is no allergic response or neutralization of the protein by an antibody. That is, in a preferred embodiment, the variant APRIL protein does not contain additional or different epitopes from the wild type or naturally occurring APRIL. By “epitope” or “determinant” herein is meant a portion of a protein that will generate and/or bind an antibody. Thus, in most instances, no significant amounts of antibodies are generated to a variant APRIL protein. In general, this is accomplished by not significantly altering surface residues, as outlined below nor by adding any amino acid residues on the surface which can become glycosylated, as novel glycosylation can result in an immune response, nor by the introduction of new MHC binding epitopes.

The variant APRIL proteins of the present invention may be shorter or longer than the amino acid sequences shown in FIG. 3. As used in this invention, “wild-type APRIL” is a native mammalian protein (preferably human). APRIL may be polymorphic. Thus, in a preferred embodiment, included within the definition of variant APRIL proteins are portions or fragments of the sequences depicted herein. Fragments of variant APRIIL proteins are considered variant APRIL proteins if a) they share at least one antigenic epitope; b) have at least the indicated homology; c) and preferably have variant APRIL or BAFF biological activity as defined herein.

In a preferred embodiment, as is more fully outlined below, the variant APRIL proteins include further amino acid variations, as compared to a wild-type APRIL, than those outlined herein. Examples include, but are not limited to, amino acid substitutions introduced to enable soluble expression in E. coli, amino acid substitutions introduced to optimize solution behavior, and amino acid substitutions introduced to modulate immunogenicity. In addition, as outlined herein, any of the variations depicted herein may be combined in any way to form additional novel variant APRIL proteins.

In addition, variant APRIL proteins may be made that are longer than those depicted in the figures, for example, by the addition of epitope or purification tags, as outlined herein, the addition of other fusion sequences, etc. For example, the variant APRIL proteins of the invention may be fused to other therapeutic proteins or to other proteins such as Fc or serum albumin for pharmacokinetic purposes. See for example U.S. Pat. Nos. 5,766,883 and 5,876,969, both of which are expressly incorporated by reference.

Variant APRIL proteins may also be identified as being encoded by variant APRIL nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred.

In a preferred embodiment, a variant APRIL nucleic acid encodes a variant APRIL protein. As will be appreciated by those in the art, due to the degeneracy of the genetic code, an extremely large number of nucleic acids may be made, all of which encode the variant APRIL proteins of the present invention. Thus, having identified a particular amino acid sequence, those skilled in the art could make any number of different nucleic acids, by simply modifying the sequence of one or more codons in a way that does not change the amino acid sequence of the variant APRIL.

The variant APRIL proteins and nucleic acids of the present invention are recombinant. As used herein, “nucleic acid” may refer to either DNA or RNA, or molecules that contain both deoxy-and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated variant APRIL nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild-type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a variant APRIL protein from one organism in a different organism or host cell. Alternatively, the protein may be made at a significantly higher concentration than is normally seen, through the use of an inducible promoter or high expression promoter, such that the protein is made at increased concentration levels. Furthermore, all of the variant APRIL proteins outlined herein are in a form not normally found in nature, as they contain amino acid substitutions, insertions and deletions, with substitutions being preferred.

Also included within the definition of variant APRIL proteins of the present invention are amino acid sequence variants of the variant APRIL sequences outlined herein. That is, the variant APRIIL proteins may contain additional variable positions as compared to human APRIL. These variants fall into one or more of three classes: substitutional, insertional or deletional variants. These variants ordinarily are prepared by site-specific mutagenesis of nucleotides in the DNA encoding a variant APRIL protein, using cassette or PCR mutagenesis or other techniques well known in the art, to produce DNA encoding the variant, and thereafter expressing the DNA in recombinant cell culture as outlined above. However, variant APRIL protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the variant APRIL protein amino acid sequence. The variants typically exhibit the same qualitative biological activity as the naturally occurring analogue; although variants can also be selected which have modified characteristics.

While the site or region for introducing an amino acid sequence variation is predetermined, the mutation per se need not be predetermined. For example, in order to optimize the performance of a mutation at a given site, random mutagenesis may be conducted at the target codon or region and the expressed variant APRIL proteins screened for the optimal combination of desired activity. Techniques for making substitution mutations at predetermined sites in DNA having a known sequence are well known, for example, M13 primer mutagenesis and PCR mutagenesis. Screening of the mutants is done using assays of variant APRIL protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final molecule. Generally these changes are done on a few amino acids to minimize the alteration of the molecule. However, larger changes may be tolerated in certain circumstances.

The variants typically exhibit the same qualitative biological activity and will elicit the same immune response as the original variant APRIL protein, although variants also are selected to modify the characteristics of the variant APRIL proteins as needed. Alternatively, the variant may be designed such that the biological activity of the variant APRIL protein is altered. For example, glycosylation or phosphorylation sites may be altered or removed. Similarly, the biological function may be altered; for example, in some instances it may be desirable to have more or less potent APRIL activity.

The variant APRIL proteins and nucleic acids of the invention can be made in a number of ways. Individual nucleic acids and proteins can be made as known in the art and outlined below. Alternatively, libraries of variant APRIIL proteins can be made for testing. In a preferred embodiment, sets or libraries of variant APRIL proteins may be generated in many ways known to those skilled in the art.

In a preferred embodiment, the different protein members of the variant APRIL library may be chemically synthesized. This is particularly useful when the designed proteins are short, preferably less than 150 amino acids in length, with less than 100 amino acids being preferred, and less than 50 amino acids being particularly preferred, although as is known in the art, longer proteins may be made chemically or enzymatically. See for example Wilken et al, Curr. Opin. Biotechnol. 9:412-26 (1998), hereby expressly incorporated by reference.

In a preferred embodiment, particularly for longer proteins or proteins for which large samples are desired, the library sequences are used to create nucleic acids such as DNA which encode the member sequences and which may then be cloned into host cells, expressed and assayed, if desired. Thus, nucleic acids, and particularly DNA, may be made which encodes each member protein sequence. This is done using well-known procedures. The choice of codons, suitable expression vectors and suitable host cells will vary depending on a number of factors, and may be easily optimized as needed.

In a preferred embodiment, multiple PCR reactions with pooled oligonucleotides are done. In this embodiment, overlapping oligonucleotides are synthesized which correspond to the full-length gene. Again, these oligonucleotides may represent all of the different amino acids at each variant position or subsets. In a preferred embodiment, these oligonucleotides are pooled in equal proportions and multiple PCR reactions are performed to create full-length sequences containing the combinations of mutations defined by the library. In addition, this may be done using error-prone PCR methods.

In a preferred embodiment, the different oligonucleotides are added in relative amounts corresponding to the probability distribution table. The multiple PCR reactions thus result in full-length sequences with the desired combinations of mutations in the desired proportions.

In a preferred embodiment, each overlapping oligonucleotide comprises only one position to be varied; in alternate embodiments, the variant positions are too close together to allow this and multiple variants per oligonucleotide are used to allow complete recombination of all the possibilities. That is, each oligo may contain the codon for a single position being mutated, or for more than one position being mutated. The multiple positions being mutated must be close in sequence to prevent the oligo length from being impractical. For multiple mutating positions on an oligonucleotide, particular combinations of mutations may be included or excluded in the library by including or excluding the oligonucleotide encoding that combination. For example, as discussed herein, there may be correlations between variable regions; that is, when position X is a certain residue, position Y must (or must not) be a particular residue. These sets of variable positions are sometimes referred to herein as a “cluster”. When the clusters are comprised of residues close together, and thus can reside on one oligonucleotide primer, the clusters can be set to the “good” correlations, and eliminate the bad combinations that may decrease the effectiveness of the library. However, if the residues of the cluster are far apart in sequence, and thus will reside on different oligonucleotides for synthesis, it may be desirable to either set the residues to the “good” correlation, or eliminate them as variable residues entirely. In an alternative embodiment, the library may be generated in several steps, so that the cluster mutations only appear together. This procedure, i.e. the procedure of identifying mutation clusters and either placing them on the same oligonucleotides or eliminating them from the library or library generation in several steps preserving clusters, can considerably enrich the experimental library with properly folded protein. Identification of clusters may be carried out by a number of ways, e.g. by using known pattern recognition methods, comparisons of frequencies of occurrence of mutations or by using energy analysis of the sequences to be experimentally generated (for example, if the energy of interaction is high, the positions are correlated). These correlations may be positional correlations (e.g. variable positions 1 and 2 always change together or never change together) or sequence correlations (e.g. if there is residue A at position 1, there is always residue B at position 2). See: Pattern discovery in Biomolecular Data: Tools, Techniques, and Applications; edited by Jason T. L. Wang, Bruce A. Shapiro, Dennis Shasha. New York: Oxford University, 1999; Andrews, Harry C. Introduction to mathematical techniques in pattern recognition; New York, Wiley-Interscience [1972]; Applications of Pattern Recognition; Editor, K. S. Fu. Boca Raton, Fla. CRC Press, 1982; Genetic Algorithms for Pattern Recognition; edited by Sankar K. Pal, Paul P. Wang. Boca Raton: CRC Press, c1996; Pandya, Abhijit S., Pattern recognition with neural networks in C++/Abhijit S. Pandya, Robert B. Macy. Boca Raton, Fla.: CRC Press, 1996; Handbook of pattern recognition & computer vision/edited by C. H. Chen, L. F. Pau, P. S. P. Wang. 2nd ed. Singapore; River Edge, N. J.: World Scientific, c1999; Friedman, Introduction to Pattern Recognition: Statistical, Structural, Neural, and Fuzzy Logic Approaches; River Edge, N. J.: World Scientific, c1999, Series title: Series in machine perception and artificial intelligence; vol. 32; all of which are expressly incorporated by reference. In addition, programs used to search for consensus motifs can be used as well.

Oligonucleotides with insertions or deletions of codons may be used to create a library expressing different length proteins. In particular computational sequence screening for insertions or deletions may result in secondary libraries defining different length proteins, which can be expressed by a library of pooled oligonucleotide of different lengths.

In another preferred embodiment, variant APRIL proteins of the invention are created by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are expressly incorporated by reference in their entirety. This set of sequences may also be an artificial set; for example, from a probability table (for example generated using SCMF) or a Monte Carlo set. Similarly, the “family” can be the top 10 and the bottom 10 sequences, the top 100 sequences, etc. This may also be done using error-prone PCR.

Thus, in a preferred embodiment, in silico shuffling is done using the computational methods described herein. That is, starting with two libraries or two sequences, random recombinations of the sequences may be generated and evaluated.

In a preferred embodiment variant APRIL proteins are chimeras formed from two or more naturally occurring APRIL or BAFF proteins. In a particularly preferred embodiment, the chimeras are formed by joining one or more receptor contact region from one or more naturally occurring APRIL proteins with the amino acid sequence of another naturally occurring APRIL or BAFF protein.

In a preferred embodiment, error-prone PCR is done to generate a library of variant APRIL proteins. See U.S. Pat. Nos. 5,605,793, 5,811,238, and 5,830,721, all of which are hereby incorporated by reference. This may be done on the optimal sequence or on top members of the library, or some other artificial set or family. In this embodiment, the gene for the optimal sequence found in the computational screen of the primary library may be synthesized. Error-prone PCR is then performed on the optimal sequence gene in the presence of oligonucleotides that code for the mutations at the variant positions of the library (bias oligonucleotides). The addition of the oligonucleotides will create a bias favoring the incorporation of the mutations in the library. Alternatively, only oligonucleotides for certain mutations may be used to bias the library.

In a preferred embodiment, gene shuffling with error-prone PCR can be performed on the gene for the optimal sequence, in the presence of bias oligonucleotides, to create a DNA sequence library that reflects the proportion of the mutations found in the variant APRIL library. The choice of the bias oligonucleotides can be done in a variety of ways; they can chosen on the basis of their frequency, i.e. oligonucleotides encoding high mutational frequency positions can be used; alternatively, oligonucleotides containing the most variable positions can be used, such that the diversity is increased; if the secondary library is ranked, some number of top scoring positions may be used to generate bias oligonucleotides; random positions may be chosen; a few top scoring and a few low scoring ones may be chosen; etc. What is important is to generate new sequences based on preferred variable positions and sequences.

In a preferred embodiment, PCR using a wild-type gene or other gene may be used, as is schematically depicted in the Figures. In this embodiment, a starting gene is used; generally, although this is not required, the gene is usually the wild-type gene. In some cases it may be the gene encoding the global optimized sequence, or any other sequence of the list, or a consensus sequence obtained e.g. from aligning homologous sequences from different organisms. In this embodiment, oligonucleotides are used that correspond to the variant positions and contain the different amino acids of the library. PCR is done using PCR primers at the termini, as is known in the art. This provides two benefits. First, this generally requires fewer oligonucleotides and may result in fewer errors. Second, it has experimental advantages in that if the wild-type gene is used, it need not be synthesized.

Using the nucleic acids of the present invention, which encode a variant APRIL protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the variant APRIL protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

In a preferred embodiment, when the endogenous secretory sequence leads to a low level of secretion of the naturally occurring protein or of the variant APRIL protein, a replacement of the naturally occurring secretory leader sequence is desired. In this embodiment, an unrelated secretory leader sequence is operably linked to a variant APRIL encoding nucleic acid leading to increased protein secretion. Thus, any secretory leader sequence resulting in enhanced secretion of the variant APRIL protein, when compared to the secretion of APRIL and its secretory sequence, is desired. Suitable secretory leader sequences that lead to the secretion of a protein are known in the art.

In another preferred embodiment, a secretory leader sequence of a naturally occurring protein or a protein is removed by techniques known in the art and subsequent expression results in intracellular accumulation of the recombinant protein.

Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the fusion protein; for example, transcriptional and translational regulatory nucleic acid sequences from Bacillus are preferably used to express the fusion protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in cells, particularly mammalian cells, such as the CMV promoter, particularly in combination with a Tet regulatory element.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

A preferred expression vector system is a retroviral vector system such as is generally described in PCT/US97/01019 and PCT/US97/01048, both of which are hereby expressly incorporated by reference.

In a preferred embodiment, the expression vector comprises the components described above and a gene encoding a variant APRIL protein. As will be appreciated by those in the art, all combinations are possible and accordingly, as used herein, the combination of components, comprised by one or more vectors, which may be retroviral or not, is referred to herein as a “vector composition”.

The variant APRIL nucleic acids are introduced into the cells either alone or in combination with an expression vector. By “introduced into ” or grammatical equivalents herein is meant that the nucleic acids enter the cells in a manner suitable for subsequent expression of the nucleic acid. The method of introduction is largely dictated by the targeted cell type, discussed below. Exemplary methods include CaPO₄ precipitation, liposome fusion, lipofectin®, electroporation, viral infection, etc. The variant APRIL nucleic acids may stably integrate into the genome of the host cell (for example, with retroviral introduction, outlined below), or may exist either transiently or stably in the cytoplasm (i.e. through the use of traditional plasmids, utilizing standard regulatory sequences, selection markers, etc.).

The variant APRIL proteins of the present invention are produced by culturing a host cell transformed with an expression vector containing nucleic acid encoding a variant APRIL protein, under the appropriate conditions to induce or cause expression of the variant APRIL protein. The conditions appropriate for variant APRIL protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Drosophila melangaster cells, Saccharomyces cerevisiae and other yeasts, E. coli, Bacillus subtilis, SF9 cells, C129 cells, 293 cells, Neurospora, BHK, CHO, COS, Pichia pastoris, etc.

In a preferred embodiment, the variant APRIL proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for the fusion protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Alternatively, Kozak sequences, signal sequences and selectable markers may be used to modulate the expression properties.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei. As outlined herein, a particularly preferred method utilizes retroviral infection, as outlined in PCT US97/01019, incorporated by reference.

As will be appreciated by those in the art, the type of mammalian cells used in the present invention can vary widely. Basically, any mammalian cells may be used, with mouse, rat, primate and human cells being particularly preferred, although as will be appreciated by those in the art, modifications of the system by pseudotyping allows all eukaryotic cells to be used, preferably higher eukaryotes. As is more fully described below, a screen will be set up such that the cells exhibit a selectable phenotype in the presence of a bioactive peptide. As is more fully described below, cell types implicated in a wide variety of disease conditions are particularly useful, so long as a suitable screen may be designed to allow the selection of cells that exhibit an altered phenotype as a consequence of the presence of a peptide within the cell.

Accordingly, suitable cell types include, but are not limited to, tumor cells of all types (particularly melanoma, myeloid leukemia, carcinomas of the lung, breast, lymphoma, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoietic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

In one embodiment, the cells may be additionally genetically engineered, that is, contain exogenous nucleic acid other than the variant APRIL nucleic acid.

The generation of variant APRIL proteins may also be generated in stable cell lines, as is well known in the art.

In a preferred embodiment, the variant APRIL proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of the variant APRIL protein into mRNA. A bacterial promoter has a transcription initiation region that is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter may include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the variant APRIL protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). For expression in bacteria, usually bacterial secretory leader sequences, operably linked to a variant APRIL encoding nucleic acid, are preferred. The bacterial expression vector may also include a selectable marker gene to allow for the selection of bacterial strains that have been transformed. Suitable selection genes include genes that render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, variant APRIL proteins are produced in insect cells. Expression vectors for the transformation of insect cells, and in particular, baculovirus-based expression vectors, are well known in the art.

In another preferred embodiment, variant APRIL protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1, 10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

In a preferred embodiment, modified APRIL variants are covalently coupled to at least one additional APRIL or BAFF variant via a linker to improve the dominant negative action of the modified domains. A number of strategies may be used to covalently link modified receptor domains together. These include, but are not limited to, linkers, such as polypeptide linkages between N- and C-termini of two domains, linkage via a disulfide bond between monomers, and linkage via chemical cross-linking reagents. Alternatively, the N- and C-termini may be covalently joined by deletion of portions of the N- and/or C-termini and linking the remaining fragments via a linker or linking the fragments directly.

By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. In one aspect of this embodiment, the linker is a peptide bond. Choosing a suitable linker for a specific case where two polypeptide chains are to be connected depends on various parameters, e.g., the nature of the two polypeptide chains, e.g., whether they naturally oligomerize (e.g., form a dimer or not), the distance between the N- and the C-termini to be connected if known from three-dimensional structure determination, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. These linked APRIL proteins have constrained hydrodynamic properties, that is, they form constitutive dimmers, and thus efficiently interact with other naturally occurring APRIL proteins to form a dominant negative heterotrimer or a competitive inhibitor or an agonist.

The linker peptide should have a length that is adequate to link two APRIL variant monomers in such a way that they assume the correct conformation relative to one another so that they retain the desired activity as antagonists of the native APRIL protein. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. See also WO 01/25277, incorporated herein by reference in its entirety.

In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains.

Useful linkers include glycine-serine polymers (including, for example, (GS)n, (GSGGS)n (GGGGS)n and (GGGS)n, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies.

Suitable linkers may also be identified by screening databases of known three-dimensional structures for naturally occurring motifs that can bridge the gap between two polypeptide chains. Another way of obtaining a suitable linker is by optimizing a simple linker, e.g., (Gly4Ser)n, through random mutagenesis. Alternatively, once a suitable polypeptide linker is defined, additional linker polypeptides can be created by application of PDA® technology to select amino acids that more optimally interact with the domains being linked. Other types of linkers that may be used in the present invention include artificial polypeptide linkers and inteins. In another preferred embodiment, disulfide bonds are designed to link the two APRIL monomers at inter-monomer contact sites. In one aspect of this embodiment the two monomers are linked at distances<5 Angstroms. In addition, the variant APRIL polypeptides of the invention may be further fused to other proteins, if desired, for example to increase expression or stabilize the protein.

In one embodiment, the variant APRIL nucleic acids, proteins and antibodies of the invention are labeled. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

In an alternative embodiment, APRIL variants are designed using the computational techniques described above. In this alternative embodiment, non-naturally occurring APRIL monomer or dimer variants are generated to bind to the receptor. More preferably, these variants preferably bind to the receptor and receptor antagonize (or competitively inhibit) naturally occurring TNFSF molecules to bind to the receptor. The dimer variants are more preferred as they substantially bind to the receptor interface. Preferred examples of these variants are modified at APRIL positions 143, 144, 146, 148, 172, 174, 192, 194, 196, 198, 200, 206, 207, 228, 230-237, 240-250, 271, 273, 275, 276, 278, 282, 284 and 285.

Once made, the variant APRIL proteins may be modified. Covalent and non-covalent modifications of the protein are included within the scope of the present invention. Such modifications may be introduced into a variant APRIL polypeptide by reacting targeted amino acid residues of the polypeptide with an organic derivatizing agent that is capable of reacting with selected side chains or terminal residues.

One type of covalent modification includes reacting targeted amino acid residues of a variant APRIL polypeptide with an organic derivatizing agent that is capable of reacting with selected side chains or the N-or C-terminal residues of a variant APRIL polypeptide. Derivatization with bifunctional agents is useful, for instance, for cross linking a variant APRIL protein to a water-insoluble support matrix or surface for use in the method for purifying anti-variant APRIL antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)dithio] propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the variant APRIL polypeptide included within the scope of this invention comprises altering the native glycosylation pattern of the polypeptide. “Altering the native glycosylation pattern” is intended for purposes herein to mean deleting one or more carbohydrate moieties found in native sequence variant APRIL polypeptide, and/or adding one or more glycosylation sites that are not present in the native sequence variant APRIL polypeptide.

Addition of glycosylation or phosphorylation sites to variant APRIL polypeptides may be accomplished by altering the amino acid sequence thereof. The alteration may be made, for example, by the addition of, or substitution by, one or more serine or threonine residues to the native sequence or variant APRIL polypeptide (for O-linked glycosylation sites). The variant APRIL amino acid sequence may optionally be altered through changes at the DNA level, particularly by mutating the DNA encoding the variant APRIL polypeptide at preselected bases such that codons are generated that will translate into the desired amino acids.

Addition of N-linked glycosylation sites to variant APRIL polypeptides may be accomplished by altering the amino acid sequence thereof. The alteration may be made, for example, by the addition of, or substitution by, one or more asparagine residues to the native sequence or variant APRIL polypeptide. The modification may be made for example by the incorporation of a canonical N-linked glycosylation site, including but not limited to, N-X-Y, where X is any amino acid except for proline and Y is preferably threonine, serine or cysteine. Another means of increasing the number of carbohydrate moieties on the variant APRIL polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the variant APRIL polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Such derivatized moieties may improve the solubility, absorption, and permeability across the blood brain barrier biological half-life, and the like. Such moieties or modifications of variant APRIL polypeptides may alternatively eliminate or attenuate any possible undesirable side effect of the protein and the like. Moieties capable of mediating such effects are disclosed, for example, in Remington's Pharmaceutical Sciences, 16th ed., Mack Publishing Co., Easton, Pa. (1980).

Another type of covalent modification of variant APRIL comprises linking the variant APRIL polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192; 4,179,337; 5,183,550. These nonproteinaceous polymers may also be used to enhance the variant APRIL's ability to disrupt receptor binding, and/or in vivo stability.

In another preferred embodiment, cysteines are designed into variant or wild type APRIL in order to incorporate (a) labeling sites for characterization and (b) incorporate polymerization (e.g., PEGylation) sites. By analogy to BAFF, in a preferred embodiment, APRIL is chemically modified at a position selected from the following: 131, 132, 146, 151, 160, 175, 190, 233 and 235. Rational PEGylation may be used to determine optimal positions, size of polymer, type of modification of the protein to attach the PEG molecule. This approach is described in U.S. Ser. No. 60/459,094 filed Mar. 31, 2003 and U.S. Ser. No. ______, filed Mar. 31, 2004, entitled METHODS FOR RATIONAL PEGYLATION OF PROTEINS, both hereby incorporated by reference in their entirety.

For example, labels that may be used are well known in the art and include but are not limited to biotin, tag and fluorescent labels (e.g. fluorescein). These labels may be used in various assays as are also well known in the art to achieve characterization.

A variety of coupling chemistries may be used to achieve polymerization including PEGylation, as is well known in the art. Examples include but are not limited to, the technologies of Shearwater and Enzon, which allow modification at primary amines, including but not limited to, lysine groups and the N- terminus. See, Kinstler et al, Advanced Drug Deliveries Reviews, 54, 477-485 (2002) and M J Roberts et al, Advanced Drug Delivery Reviews, 54, 459-476 (2002), both hereby incorporated by reference. Other techniques include coupling to cysteine residues via maleiimide functional moiety, either at native or engineered cysteines.

Other modifications may be made to the variant APRIL proteins of the present invention, including modifications to the protein that enhance stability, dosage administration (e.g., amphiphilic polymers, see WO 0141812A2, commercially available from Nobex Corporation), clearance (e.g., PEG, aliphatic moieties that effect binding to HSA), and the like.

Optimal sites for modification can be chosen using a variety of criteria, including but not limited to, visual inspection, structural analysis, sequence analysis and molecular simulation. Individual residues may be analyzed to identify mutational sites that will not disrupt the monomer structure. Then the distance from each side chain of a monomer to another subunit may be calculated to ensure that chemical modification will not disrupt oligomerization. It is possible that receptor binding disruption may occur and may be beneficial to the activity of the APRIL variants of this invention.

In another preferred embodiment, portions of either the N- or C-termini of the wild-type APRIL monomer are deleted while still allowing the APRIL molecule to fold properly. In addition, these modified APRIL proteins would substantially lack receptor binding and/or activation, and could optionally interact with other wild-type APRIL or BAFF molecules or modified APRIL or BAFF proteins to form trimers (or other oligomers) as described above.

More specifically, removal or deletion of from about 1 to about 55 amino acids from either the N or C termini of the extracellular domain of APRIIL, or both, are preferred. A more preferred embodiment includes deletions of N-termini beyond residue 10 and more preferably, deletion of the first 47 N-terminal amino acids. The deletion of C-terminal leucine is an alternative embodiment.

In another preferred embodiment, the wild type APRIL or variants generated by the invention may be circularly permuted. All natural proteins have an amino acid sequence beginning with an N-terminus and ending with a C-terminus. The N- and C-termini may be joined to create a cyclized or circularly permutated APRIL proteins while retaining or improving biological properties (e.g., such as enhanced stability and activity) as compared to the wild-type protein. In the case of an APRIL protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar or enhanced relative to those of the original protein. The circularly permuted APRIL protein may be further chemically modified (e.g., PEGylated or glycosylated). In a further preferred embodiment PDA® technology may be used to further optimize the APRIL variant, particularly in the regions created by circular permutation. These include the novel N- and C-termini, as well as the original termini and linker peptide.

Various techniques may be used to permutate proteins. See U.S. Pat. No. 5,981,200; Maki K, Iwakura M., Seikagaku. 2001 Jan; 73(1): 42-6; Pan T., Methods Enzymol. 2000; 317:313-30; Heinemann U, Hahn M., Prog Biophys Mol Biol. 1995; 64(2-3): 121-43; Harris M E, Pace N R, Mol Biol Rep. 1995-96; 22(2-3):115-23; Pan T, Uhlenbeck O C., 1993 Mar 30; 125(2): 111-4; Nardulli A M, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5, 1290-1300 (1996); all hereby incorporated by reference.

In addition, a completely cyclic APRIL may be generated, wherein the protein contains no termini. This is accomplished utilizing intein technology. Thus, peptides can be cyclized and in particular inteins may be utilized to accomplish the cyclization.

Cyclization and circular permutation may be used to generate the dominant-negative activity, the competitive inhibition or the agonist activity of the APRIL proteins of the present invention.

Variant APRIL polypeptides of the present invention may also be modified in a way to form chimeric molecules comprising a variant APRIL polypeptide fused to another, heterologous polypeptide or amino acid sequence. In one embodiment, such a chimeric molecule comprises a fusion of a variant APRIL polypeptide with a tag polypeptide that provides an epitope to which an anti-tag antibody can selectively bind. The epitope tag is generally placed at the amino-or carboxyl-terminus of the variant APRIL polypeptide. The presence of such epitope-tagged forms of a variant APRIL polypeptide can be detected using an antibody against the tag polypeptide. Also, provision of the epitope tag enables the variant APRIL polypeptide to be readily purified by affinity purification using an anti-tag antibody or another type of affinity matrix that binds to the epitope tag. In an alternative embodiment, the chimeric molecule may comprise a fusion of a variant APRIIL polypeptide with an immunoglobulin or a particular region of an immunoglobulin. For a bivalent form of the chimeric molecule, such a fusion could be to the Fc or Fab region of an IgG molecule. Other fusion entities include human serum albumin (HSA), hydrophilic peptides, fatty acid molecules, etc.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6): 547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

In a preferred embodiment, the variant APRIL protein is purified or isolated after expression. Variant APRIL proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the variant APRIL protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the variant APRIL protein. In some instances no purification will be necessary.

Once made, the variant APRIL proteins and nucleic acids of the invention find use in a number of applications. In a preferred embodiment, the variant APRIL proteins are administered to a patient to treat an APRIL or BAFF-related disorder.

By “BAFF or APRIL related disorder” or “BAFF or APRIL responsive disorder” or “condition” herein is meant a disorder that may be ameliorated by the administration of a pharmaceutical composition comprising a variant APRIL protein, including, but not limited to, autoimmune, inflammatory, immunological and oncological disorders. The variant APRIL proteins are major effectors in the pathogenesis of immune-regulated diseases.

In a preferred embodiment, the variant APRIL protein is used to treat for example, congestive heart failure (CHF), vasculitis, rosecea, acne, exzcema, myocarditis and other conditions of the myocardium, systemic lupus erythematosus, diabetes, spondylopathies, synovial fibroblasts, and bone marrow stroma; bone loss; Padgett's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; multiple myeloma; osteolytic bone cancers, breast cancer, lung cancer, kidney cancer and rectal cancer; bone metastasis, bone pain management, and humoral malignant hypercalcemia, ankylosing spondylitisa and other spondyloarthropathies; transplantation rejection, viral infections, hematologic neoplasisas and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemagiopericytoma), osteoporosis, hepatitis, HIV, AIDS, spondyloarthritis, rheumatoid arthritis, inflammatory bowel diseases (IBD), sepsis and septic shock, Crohn's Disease, psoriasis, schleraderma, graft versus host disease (GVHD), allogenic islet graft rejection, hematologic malignancies, such as multiple myeloma (MM), myelodysplastic syndrome (MDS) and acute myelogenous leukemia (AML), cancer and the inflammation associated with tumors, peripheral nerve injury or demyelinating diseases.

By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 0.01 to about 50 μg/kg are used, administered either intravenously, subcutaneously, intratracheally or orally. As is known in the art, adjustments for variant APRIL protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

The term “treatment” in the instant invention is meant to include therapeutic treatment, as well as prophylactic, or suppressive measures for the disease or disorder. Thus, for example, successful administration of a variant APRIL protein prior to onset of the disease results in “treatment” of the disease. As another example, successful administration of a variant APRIL protein after clinical manifestation of the disease to combat the symptoms of the disease comprises “treatment” of the disease. “Treatment” also encompasses administration of a variant APRIL protein after the appearance of the disease in order to eradicate the disease. Successful administration of an agent after onset and after clinical symptoms have developed, with possible abatement of clinical symptoms and perhaps amelioration of the disease, comprises “treatment” of the disease.

Those “in need of treatment” include mammals already having the disease or disorder, as well as those prone to having the disease or disorder, including those in which the disease or disorder is to be prevented.

In another embodiment, a therapeutically effective dose of a variant APRIL protein, a variant APRIL gene, or a variant APRIL antibody is administered to a patient having a disease involving inappropriate expression of an APRIL or BAFF protein. A “disease involving inappropriate expression of a BAFF or APRIL protein” within the scope of the present invention is meant to include diseases or disorders characterized by aberrant APRIL or BAFF proteins, either by alterations in the amount of BAFF or APRIL protein present or due to the presence of mutant BAFF or APRIL protein. An overabundance may be due to any cause, including, but not limited to, overexpression at the molecular level, prolonged or accumulated appearance at the site of action, or increased activity of BAFF or APRIL protein relative to normal. Included within this definition are diseases or disorders characterized by a reduction of BAFF or APRIL protein. This reduction may be due to any cause, including, but not limited to, reduced expression at the molecular level, shortened or reduced appearance at the site of action, mutant forms of BAFF or APRIL protein, or decreased activity of BAFF or APRIL protein relative to normal. Such an overabundance or reduction of BAFF or APRIL protein can be measured relative to normal expression, appearance, or activity of BAFF or APRIL protein according to, but not limited to, the assays described and referenced herein.

The administration of the variant APRIL proteins of the present invention, preferably in the form of a sterile aqueous solution, may be done in a variety of ways, including, but not limited to, orally, subcutaneously, intravenously, intranasally, intraotically, transdermally, topically (e.g., gels, salves, lotions, creams, etc.), intraperitoneally, intramuscularly, intrapulmonary (e.g., AERx® inhalable technology commercially available from Aradigm or Inhance™ pulmonary delivery system commercially available from Inhale Therapeutics), vaginally, rectally, or intraocularly. In some instances, for example, in the treatment of wounds, inflammation, etc., the variant APRIL protein may be directly applied as a solution or spray. Depending upon the manner of introduction, the pharmaceutical composition may be formulated in a variety of ways.

Also, sustained release or controlled release formulations may be used for the compositions of the present invention. For example, ProLease® (commercially available from Alkermes) a microsphere-based delivery system composed of the desired bioactive molecule incorporated into a matrix of poly-DL-lactide-co-glycolide (PLG) and other pharmaceutically compatible polymeric matrices may be used to create sustained release formulations.

The concentration of the therapeutically active variant APRIL protein in the formulation may vary from about 0.1 to 100 weight %. In another preferred embodiment, the concentration of the variant APRIL protein is in the range of 0.003 to 1.0 molar, with dosages from 0.03, 0.05, 0.1, 0.2, and 0.3 millimoles per kilogram of body weight being preferred.

The pharmaceutical compositions of the present invention comprise a variant APRIL protein in a form suitable for administration to a patient. In the preferred embodiment, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant APRIL proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

In one embodiment provided herein, antibodies, including but not limited to monoclonal and polyclonal antibodies, are raised against variant APRIL proteins using methods known in the art. In a preferred embodiment, these anti-variant APRIL antibodies are used for immunotherapy. Thus, methods of immunotherapy are provided. By “immunotherapy” is meant treatment of BAFF or APRIL-related disorders with an antibody raised against a variant APRIL protein. As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with a variant APRIL protein antigen to which antibodies are raised. As appreciated by one of ordinary skill in the art, the variant APRIL protein antigen may be provided by injecting a variant APRIL polypeptide against which antibodies are desired to be raised into a recipient, or contacting the recipient with a variant APRIL protein encoding nucleic acid, capable of expressing the variant APRIL protein antigen, under conditions for expression of the variant APRIL protein antigen.

In another preferred embodiment, a therapeutic compound is conjugated to an antibody, preferably an anti-variant APRIL protein antibody. The therapeutic compound may be a cytotoxic agent. In this method, targeting the cytotoxic agent to tumor tissue or cells, results in a reduction in the number of afflicted cells, thereby reducing symptoms associated with cancer, and variant BAFF or APRIL protein related disorders. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against cell cycle proteins, or binding of a radionuclide to a chelating agent that has been covalently attached to the antibody.

In a preferred embodiment, variant APRIL proteins are administered as therapeutic agents, and can be formulated as outlined above. Similarly, variant APRIL genes (including both the full-length sequence, partial sequences, or regulatory sequences of the variant APRIL coding regions) may be administered in gene therapy applications, as is known in the art. These variant APRIL genes may include antisense applications, either as gene therapy (i.e. for incorporation into the genome) or as antisense compositions, as will be appreciated by those in the art.

In a preferred embodiment, the nucleic acid encoding the variant APRIL proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy, where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. [Zamecnik et al., Proc. Natl. Acad. Sci. U.S.A. 83:4143-4146 (1986)]. The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection [Dzau et al., Trends in Biotechnology 11:205-210 (1993)]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

In another embodiment, variant APRIL genes are administered as DNA vaccines, either single genes or combinations of variant APRIL or BAFF genes. Naked DNA vaccines are generally known in the art. Brower, Nature Biotechnology, 16:1304-1305 (1998). Methods for the use of genes as DNA vaccines are well known to one of ordinary skill in the art, and include placing a variant APRIL gene or portion of a variant APRIL gene under the control of a promoter for expression in a patient in need of treatment.

The variant APRIL gene used for DNA vaccines can encode full-length variant APRIL proteins, but more preferably encodes portions of the variant APRIL proteins including peptides derived from the variant APRIL protein. In a preferred embodiment a patient is immunized with a DNA vaccine comprising a plurality of nucleotide sequences derived from a variant APRIL gene. Similarly, it is possible to immunize a patient with a plurality of variant APRIL genes or portions thereof as defined herein. Without being bound by theory, expression of the polypeptide encoded by the DNA vaccine, cytotoxic T-cells, helper T-cells and antibodies are induced which recognize and destroy or eliminate cells expressing APRIL or BAFF proteins.

In a preferred embodiment, the DNA vaccines include a gene encoding an adjuvant molecule with the DNA vaccine. Such adjuvant molecules include cytokines that increase the immunogenic response to the variant APRIL polypeptide encoded by the DNA vaccine. Additional or alternative adjuvants are known to those of ordinary skill in the art and find use in the invention.

All references cited herein, including patents, patent applications (provisional, utility and PCT), and publications are incorporated by reference in their entirety.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### EXAMPLES

### Example 1

### APRIL Library Expression, Purification, and Activity Assays for APRIL Variants

Overnight Culture Preparation:

Competent Tuner(DE3)pLysS cells in 96 well-PCR plates are transformed with 1 ul of APRIL library DNAs and spread on LB agar plates with 34 mg/ml chloramphenicol and 100 mg/ml ampicillin. After overnight growth at 37 degrees C., a colony is picked from each plate in and inoculated into 15 ml 1.5 ml of CG media supplemented with 34 mg/ml chloramphenicol and 100 mg/ml ampicillin or carbenicillin kept in 96 deep well block. The block culture is shaken at 250 rpm at 37 degrees C. overnight.

Expression:

The next day, the overnight cultures are diluted 1:50 into 2000 ml shake flasks containing 500 ml CG media supplemented with 100 mg/ml ampicillin or carbenicillin. Cells are grown at 30C at 250 rpm until OD600=0.6-0.7 is reached at which time IPTG is added to final concentration of 1 mM. Subsequently the cultures are grown at 16C for 12 hours or overnight.

Lysis:

Cells are harvested by centrifugation in the centrifuge 0.5 L bottles at 10,000 g for 30′. The 24-well block is centrifuged at 3000 rpm for 10 minutes. The pellets are resuspended in lysis buffer (22.5 ml PBS pH 7.2, 2.5 ml 10× BugBuster, 8 ul Benzonase) and mixed gently and let stand for 10 min. at RT. The suspension is spun down at 30,000 g for 30 min. The supernatant is clarified using a 0.45 um syringe filter and kept on ice or at 4C until ready for purification.

Batch Binding and Purification Employing Anti-Flag M2 Antibody Resin:

5 ml of slurry of Anti-FLAG M2 antibody affinity resin (Sigma, #A220) is added to a BioRad Econo Pak column (20 ml) and washed with 25 ml of PBS pH=7.2. Then the resin is washed with 15 ml of 0.1 M glycine-HCl pH 3.0 and equilibrated with 25 CV PBS pH=7.2. After removal of PBS, lysed supernatant is added to the resin, the Econo Pak is capped and incubated at room temperature for 1 hr with gentle agitation. Then the Econo Pak is placed vertically and the supernatant is drained by gravity. The resin is washed with 25 ml of PBS. The remaining beads are rinsed off with 0.5 ml column buffer and transferred into a column. Protein from the resin is eluted with 10 ml of elution buffer (100 ug/ml FLAG peptide (Sigma, #F3290) diluted in PBS pH=7.2). The resin is washed with 5 CV 100 mM glycine pH 3.0.

Purification of His-APRIL Variants Employing Ni NTA Column Resin:

The pellets are resuspended in 70 ml of lysis buffer (50 mM NaH2PO4, 300 mM NaCl, 10 mM imidazole). After freezing at −80 degrees C. for 20 minutes and thawing at 37 C twice, MgCl₂ is added to 10 mM, and DNase I to 75 mg/ml. The mixture is incubated at 37 C. for 30 minutes. Purification is carried out following the Qiagen Ni NTA spin column purification protocol for native condition. The purified protein is dialyzed against 1×PBS for 1 hour at 4 degrees C. four times. Dialyzed protein is filter sterilized, using Millipore multiscreenGV filter plate to allow the addition of protein to the sterile mammalian cell culture assay later on.

APRIL Purification of Untagged and myc-Tagged Proteins

Cells are lysed in lysis buffer with sonication (25 mM Tris pH 7.5, 25 mM MgCl2, 10% glycerol) and spun at 30,000 g for 15 min at 4C. The supernatant is filtered through 0.45 um filter, passed over a QHP column (Amersham Bioscience), and eluted with a linear gradient of NaCl 0-500 mM in 20Column Volumes of elution buffer (25 mM Tris pH 7.5, 25 mM MgCl2, 10% glycerol). The fractions are analyzed by SDS-PAGE and the most concentrated fractions are pooled. The pH is lowered and diluted with the following procedure: 1. Add equal volume of 10% glycerol in milliQ water to pooled fractions; 2. Add MgCl₂ to 25 mM final concentration; 3. Decrease conductivity to <7 mS/cm with additional 10% glycerol in milliQ water; 4. Add 0.1 volume of 10× Citrate Buffer (250 mM sodium citrate at pH4.0) and 5. Filter solution. The filtered solution is passed over a Resource S column (Amersham Bioscience), eluted with a linear gradient of NaCl 0-500 mM in 20Column Volumes of elution buffer (25 mM sodium citrate pH 4.0, 25 mM MgCl2, 10% glycerol), and fractions are analyzed by SDS-PAGE. The cleanest fractions are pooled and concentrated. Then they are dialyzed into a formulation buffer (2 m mM sodium citrate, 25 mM MgCl2, 100 mM NaCl, 10% glycerol, pH=7.0).

Quantification:

Purified proteins are quantified by BCA protein assay (Pierce Biotechnology, #23227) according to the manufacturer's protocol. All measurements are performed in triplicate.

### Example 2

### In Vitro APRIL Bioassay

B Cell Purification:

The human peripheral blood mononuclear cells are obtained from leukophoresis pack of consenting donors by running through the standard ficoll gradient. The B cells are obtained from PBMC fraction by negative depletion of non-B cells from PBMC using the magnetic labeling-purification system (MACS™ technology from Miltenyi Biotec, Germany). The resulting B-cells are ready for use in either B cell proliferation/viability assay.

B cell proliferation/viability (agonist) assay using Titer-Glo Luminescent Cell Viability Assay kit (Promega, Inc., Wisconsin, USA): Cross-linking with an anti-Flag M2 mAb improves the dynamic range of the assay and results in similar or increased activity of Flag-APRILs relative to that of untagged APRIL(wt). The signal remains the same in the range of anti-Flag M2 antibody. 5,500-7,500 purified B cells in RPMI1649 (supplemented with 10% FBS) are added in 25 ul aliquots into well of 384 micro-titer plates with. Various amounts of APRIL (in the range of 100 ng/ml-0.005 ng/ml) are added to cells; 2 mg/ml of anti-m antibody is added as a co-stimulator. If APRIL preparations have Flag-tag, the M2 anti-Flag antibody is added to the constant concentration of 2 mg/ml. The plate is then incubated at 37° C. with 5% CO2 for 4 days. After 4 days of incubation, 25 ml of luminescence substrate/buffer solution of Cell Titer-Glo kit is added and the resulting luminescence was detected using a standard luminometer. The data is plotted using Prizm.

Assay specificity is confirmed by using APRIL-specific soluble commercial APRIL receptor-Fc fusions as antagonists and FlagAPRIL(wt), FlagAPRIL single variant, FlagAPRIL double variant. The converse experiment is performed using Flag-APRIL single variant: soluble APRIL/BAFF receptors are held at constant 10 mg/ml and varying amounts of Flag-APRIL single variant is added.

Donor-to-donor reproducibility is assessed by using PBMCs isolated from two different donors. Proliferation response to Flag-APRIL single variant or Flag-APRIL double variant is checked to determine if the results are similar in PBMC preparations obtained from either donor indicating that the assay is stable and reproducible.

Antagonist Assay Using Titer-Glo

Various proteins including the Fc fusions of APRIL/BAFF receptors (BAFF-R, BCMA, and/or TACI) and APRIL variants are used to antagonize the APRIL/BAFF induced cell proliferation using the above-described B cell proliferation/viability assay. The purified B cells are incubated with a fixed amount of agonist such as wild type APRIL or BAFF in the presence of variable amount of the antagonist for 4 days at 37° C. incubator with 5% CO2.

B cell proliferation (agonist) assay using Cell Proliferation ELISA, BrdU (chemiluminescence) kit, Roche Diagnostics, #1 669 915): B-cells are incubated in the presence of various concentrations (10-0.00001 ug/ml) of APRIL wt or APRIL variants in a white 96-well MTP (tissue culture grade; flat, clear bottom) at a final volume of 100 ul/well in a humidified atmosphere at 37° C. The incubation period of the microcultures depends on the particular experimental approach and on the cell type used for the assay. For most experimental setups, an incubation period of about 24 to 120 hours is appropriate.

Labeling the Cells with BrdU:

10 ul/well BrdU labeling solution is added if the cells were cultured in 100 ul/well (final concentration: 10 uM BrdU) and the cells are incubated for an additional 2 to 24 h at 37° C. (if the cells were cultured in 200 ul/well, add 20 ul/well BrdU labeling solution).

Removal of Labeling Medium:

The MTP is centrifuged at 300×g for 10 min and the labeling medium is removed by flicking off or suction using a canulla. Cells are dried using a hair-dryer for about 15 min or, alternatively, at 60° C. for 1 h.

Cell Fixation and DNA Denaturation:

200 ul/well FixDenat is added to the cells and they are incubated for 30 min at 15 25° C.

Incubation with anti-BrdU-POD:

FixDenat solution is removed thoroughly by flicking off and tapping. 100 ul/well anti-BrdU-POD working solution is added and the solution is incubated for approx. 90 min at 15-25° C. Alternatively, this incubation period may be varied between 30-120 min, depending on individual requirements.

Washing:

The antibody conjugate is removed by flicking off and the wells are rinsed three times with 200-300 ul/well washing solution.

Substrate Reaction:

The washing solution is removed by tapping, the clear bottom is sealed with black adhesive foil and 100 ul/well substrate solution is added to each well with a multi-channel pipette. Then the contents are incubated at 15-25° C. for at least 3 min on a shaker.

Measurement:

The light emission of the samples is measured in a microplate luminometer with photomultiplier technology.

### Example 3

### Binding Assay (Plate Format)

Biotinylation of APRIL is performed by adding 20 molar excess Sulfo-NHS-LC-biotin to the protein sample and incubation of the sample on ice for 2 hours. Excess biotin is removed from the sample by dialysis. The protein concentration of biotinylated APRIL is determined by BCA protein assay (Pierce). Wells of a microtiter plate are coated with anti-FLAG antibody at a concentration of 2.5 mg/ml and blocked with 3% BSA overnight at 4° C. The FLAG-tagged protein BAFF-R, TACI, or BCMA receptors are added at a concentration of 10 ng/ml in PBS=1% BSA to wells of the anti-FLAG-coated microtiter plate, and the plate is incubated for 2 hours at room temperature. Biotinylated APRIL proteins ranging in concentrations from about 0-1 mg/mL are added in quadruplicate to anti-FLAG-BAFF-R, TACI, or BCMA receptors coated wells to represent total binding. Non-specific binding is measured by adding biotinylated APRIL proteins ranging in concentrations from about 0-1 μg/ml in quadruplicate to wells coated only with anti-FLAG antibody. Binding is allowed to occur overnight at +4° C. to ensure equilibrium. Alkaline phosphatase conjugated neutravidin (Pierce) is added to the wells at 1:10,000 dilution in PBS+1% BSA and incubated for 30 min at room temperature. Luminescence is detected upon the addition of the CSPD star substrate (Applied Biosystems, Foster City, Calif.) and is measured (Wallac VICTOR, Perkin Elmer Life Sciences, Boston, Mass.). The specific binding of APRIL was calculated by subtracting non-specific binding from total binding. Data is fit to the binding equation y=(BLmax*x) /(Kd+x).

Binding Assay (Alpha Screen Format):

Biotinylation of APRIL is performed by adding 20-fold molar excess Sulfo-NHS-biotin to protein sample and incubating 30 minutes at room temperature. Excess biotin is removed from the sample by dialysis. The protein concentration of biotinylated APRIL is determined by BCA Protein assay (Pierce). APRIL variant proteins ranging in concentrations from 1 pM to 100 nM, biotinylated APRIL, one of BAFF-R/Fc, TACI/Fc, or BCMA/Fc chimera (R&D Systems), and AlphaScreen TM protein A acceptor beads (Perkin Elmer LAS), all in assay buffer [100 mM Tris (pH 8.0), 0.01% Tween-20, 0.1% BSA], are added together in a 96 well reaction plate and incubated for one hour at room temperature. AlphaScreen streptavidin donor beads (Perkin Elmer LAS) in assay buffer are added to the reactions and incubated for two hours at room temperature, then transferred to a white, 384-well plate and read in a Packard Fusion multiplatform plate reader (Perkin Elmer LAS).

The competition of APRIL variants in the binding assay is plotted in GraphPad Prism and fit to a nonlinear regression for one site competition to determine EC50 Binding properties of in house produced Flag-APRIL and 10×His-APRIL as compared to commercially available APRIL and to determine if the reaction buffer shows any apparent binding in the AlphaScreen™ assay.

Competition experiments are conducted between tested cytokine (His-APRIL, Flag-APRIL) and a soluble receptor (e.g., TACI and BCMA) to determine the binding curves by APRIL-biotin and the soluble receptor.

### Example 4

### APRIL Exchange—Native Gels

Two types of exchange reactions are tested: 1) HisAPRIL variant and FlagAPRIL, or 2) FlagAPRIL variant and untagged APRIL(wt). The reaction is monitored at 24, 72 and 168 hrs time points. A Western blot with anti-Flag and anti-His antibodies will confirm the oligomerization state. The exchange bands should be visible after 24 hrs incubation. After about a week, the reaction should reach equilibrium.

Since efficient exchange between the FlagAPRIL variant and Untagged APRIL(wt) is observed to determine if mechanisms alternative to the dissociation/re-association type of exchange should be considered.

APRIL Exchange—anisotropy assay:

In order to measure the kinetics of exchange between APRIL trimers in solution a novel spectroscopic assay was developed. This technique utilizes the polarization anisotropy differences between homotrimers of fluorescently modified APRIL and heterotrimers formed between fluorescent and unlabeled APRIL molecules. Since this assay is carried out in a real-time sampling device, the formation of APRIL heterotrimers as a function of time is measured. Furthermore, this assay is sensitive to a variety of buffers and/or excipients thereby enabling a detailed kinetic analysis of APRIL exchange in solution.

This assay necessitates a fluorescently labeled APRIL trimer that at limiting concentrations could be used as a tracer to monitor exchange. We generate a APRIL variant specifically labeled with Alexa568 maleimide. Polarization anisotropy measures heterotrimer formation at steady state. 1 ug/mL Alexa568 APRIL is mixed either alone or with increasing concentrations of RANK-L, APRIL or BAFF variants in a buffer solution for 3 days at 37C. These reactions are placed into the spectroscopic instrument and the steady-state anisotropy is measured. This experiment demonstrates the specificity (e.g., no observed exchange between Alexa568 APRIL and RANK-L) and utility of this assay (pre and post steady-state). 1 ug/mL Alexa568 APRIL is mixed together alone or with 100 ug/mL APRIL variants in a 96-well assay format and begin anisotropy measurements.

Next, the spectral properties of this modified APRIL maybe analyzed to demonstrate that it may be used to measure exchange between Alexa568 and unlabeled APRILs. Polarization anisotropy demonstrates heterotrimer formation between APRIL homotrimers. 1 ug/mL Alexa568 APRIL are mixed alone or with 0.1 ug/mL, 1 ug/mL, 3 ug/mL, 5 ug/mL, 7 ug/mL, 10 ug/mL, or 50 ug/mL APRIL variant in 96-well assay format. The plate is placed into the instrument to begin anisotropy measurements. Once the time-course is completed, the end-point samples are analyzed using native PAGE to determine the extent of Alexa568 APRIL sequestration into heterotrimers. Native PAGE analysis demonstrates that the anisotropy changes correlate perfectly with the decreased mobility of APRIL heterotrimers on these gels.

Furthermore, this assay has further utility because it is compatible with both modified (e.g., PEGylated) and unmodified cold APRILs, and it is highly specific for exchange between APRILs (e.g., APRIL fails to exchange with RANKL). Finally, other methods require either solid-phase (i.e. sandwich ELISA or RIA), or acrylamide gels (i.e. native PAGE analysis or IEF) to resolve the end products of heterotrimer formation. This assay is superior to currently utilized methods because it allows kinetic analysis in solution. The assay provides unexpected results in that measurements of a change in polarization anisotropy may be made without any apparent change in molecular weight (e.g., exchange between Alexa568 APRIL and cold variant APRIL). The experimental analysis suggests that there is an appreciable amount of fluorescent enhancement observed upon heterotrimer formation. This change may be exploited with the aid of polarizing filters to increase sensitivity and generate the anisotropy differences shown in the above examples.

The following references are incorporated by references in their entirety:
Yu, G., et al Nature Immunology 2000, 1: 252-256
Mackay, F. and Kalled, S. Current Opinion in Immunology 2002, 14:783-790
Mackay, F. and Mackay, C. TRENDS in Immunology 2002, 23:113-115
Vaux, D. J. Clin. Investigation 2002, 109:17-18
Kayagaki, N., et al. Immunity 2002, 10, 515-524
Gross, J., et al. NATURE 2000, 404:995-999
Seshasayee,, D., et al. Immunity 2003, 18: 279-288
Yan, M., et al Nature Immunology 2000, 1:37-41
Zhang, J., et al. The Journal of Immunology 2001, 166:6-10
Oren, D., et al. Nature Structural Biology 2002, 9:288-292
Moore, P., et al. Science 1999, 285:260-263
Roschke, V., et al. The Journal of Immunology 2002, 169:4314-4321
Do, R., and Chen-Kiang, C. Cytokine & Growth Factor Reviews 2002, 13:19-25
Schneider, P., et al. The Journal of Experimental Medicine 1999, 189:1747-1756
Groom, J., et al The Journal of Clinical Investigation 2002, 109:59-68
Khare, S. and Hsu, H. TRENDS in Immunology 2001, 22:61
Mackay, F. and Browning, J. Nature Reviews Immunology 2002, 2:465-475
Kanakaraj, P. et al. Cytokine, 12, 25-31 (2001)
Karpusas, M. et al. J. Mol.Biol., 315, 1145-1154 (2002)
Oren, D. A. et al. Nature Struct. Biol., 9, 288-292 (2002)
Liu, Y, et al. Nature, 423, 49-56, (2003)
Kim, H. M. et al. Nature Struct. Biol., 10, 342-348 (2003)
Liu, Y. et al. Cell, 108, 383-394 (2002)
Wu, J. and Filutowicz, M. Acta Biochim Pol. 46, 591-599 (1999)

All references cited herein are incorporated by reference in their entirety.

Whereas particular embodiments of the invention have been described above for purposes of illustration, it will be appreciated by those skilled in the art that numerous variations of the details may be made without departing from the invention as described in the appended claims. All references cited herein are expressly incorporated by reference.

1. A variant APRIL protein which modulates at least one BAFF or APRIL biological activity comprising at least one amino acid substitution at a position selected from the group consisting of:131, 132, 133, 138, 139, 140, 143, 144, 146, 148, 151, 160, 170, 171, 172, 173, 174, 175, 176, 177, 178, 179, 180, 181, 186, 187, 188, 189, 190, 192, 194, 195, 196, 197, 198, 200, 202, 205, 206, 207, 208, 228, 230, 231, 232, 233, 234, 235, 236, 237, 238, 239, 240, 241, 242, 243, 244, 245, 246, 247, 248, 249, 250, 271, 273, 275, 276, 278, 282, 284, and 285.

2. A variant APRIL protein according to claim 1, wherein:
a) at least one position is modified in an APRIL domain selected from the the group consisting of the Large Domain, Small Domain, and the DE Loop; and
b) said modification occurs at a position selected from the group consisting of 138, 139, 140, 170, 171, 172, 173, 174, 175, 176, 177, 178, 180, 181, 186, 187, 188, 189, 195, 196, 197, 198, 205, 206, 207, 208, 230, 231, 232, 233,.234, 235, 236, 237, 238, 239, and 240.

3. A variant APRIL protein according to claim 1, wherein said biological activity is selected from the group consisting of modulation of antagonist activity, modulation of BAFF receptor interactions, modulation of APRIL receptor interactions, modulation of BAFF receptor specificity and APRIL receptor specificity.

4. A variant APRIL protein according to claim 3, wherein modulation receptor binding is selected from at least one of the group consisting of: increase in BAFF-R binding, decrease in BAFF-R binding, increase in TACI binding, decrease in TACI binding, increase in BCMA binding, decrease in BCMA binding.

5. A variant APRIL protein according to claim 4, wherein said biological activity is decreased BAFF-R binding, and said substitution is selected from at least one position in the group consisting of: 131, 133, 173, 175, 176, 177, 179, 181, 192, 195, 197, 230, 231, 233, and 241.

6. A variant APRIL protein according to claim 4, wherein said biological activity is increased BAFF-R binding, and said substitution is selected from at least one position in the group consisting of: 131, 132, 133, 173, 181, 197, 202, 206, 208, 232, and 235.

7. A variant APRIL protein according to claim 4, wherein said biological activity is increased TACI binding, and said substitution is selected from at least one position in the group consisting of: 131, 132, 133, 173, 181, 197, 202, 206, 208 and 232.

8. A variant APRIL protein according to claim 4, wherein said biological activity is decreased TACI binding, and said substitution is selected from at least one position in the group consisting of: 131, 133, 173, 174, 175, 176, 177, 179, 181, 192, 195, 197, 230, 231, and 241,

9. A variant APRIL protein according to claim 4, wherein said biological activity is decreased BCMA binding, and said substitution is selected from at least one of the group consisting of: 131, 133, 173, 174, 175, 176, 177, 179, 181, 192, 195, 197, 206, 208, 230, 231, 232, 237, and 241.

10. A variant APRIL protein according to claim 4, wherein said biological activity is increased BCMA binding, and said substitution is selected from at least one of the group consisting of: 131, 132, 133, 173, 176, 181, 192, 197, 202, 206, 208, and 239.

11. A variant APRIL protein according to claim 4, wherein said antagonist activity is dominant negative antagonism or receptor antagonism.

12. A variant APRIL protein according to claim 11, wherein at least one position is selected from the group consisting of: 143, 144, 146, 148, 172, 174, 192, 194, 196, 198, 200, 206, 207, 228, 230-237, 240-250, 271, 273, 275, 276, 278, 282, 284, and 285.

13. A variant APRIL protein according to claim 1, wherein said variant APRIL protein further comprises a chemical modification.

14. A variant APRIL protein according to claim 13, wherein said chemical modification is made at at least one of the following positions: 131, 132, 146, 151, 160, 175, 190, 233 and 235.

15. A variant APRIL protein according to claim 14, wherein said chemical modification is PEGylation.

16. A pharmaceutical composition comprising a variant APRIL protein according to claim 1 and a pharmaceutical carrier.

17. A recombinant nucleic acid encoding a variant APRIL protein of claim 1.

18. An expression vector comprising the recombinant nucleic acid of claim 17.

19. A host cell comprising the recombinant nucleic acid of claim 17.

20. A host cell comprising the expression vector of claim 18.

21. A method of producing a variant APRIL protein comprising culturing the host cell of claim 19 under conditions suitable for expression of said nucleic acid.

22. The method according to claim 21 further comprising recovering said variant APRIL protein.

23. A method of forming a mixed oligomer comprising a variant APRIL protein of claim 1 with a BAFF protein.

24. A method according to claim 23 wherein said BAFF protein is a variant BAFF protein.

25. A method for treating a BAFF or APRIL-related disorder comprising administering a variant APRIL protein according to claim 1 to a patient in need of said treatment.
